(19) United States
(12) Patent Application Publication
(10) Pub. No.: US 2022/0409627 A1
(43) Pub. Date: Dec. 29, 2022

(54) TREATMENT OF LOWER AIRWAYS DISORDERS

(71) Applicant: Altavant Sciences GmbH, Basel (CH)

(72) Inventors: Lyn A. BARANOWSKI, Brooklyn, NY (US); Brian A. LORTIE, Paoli, PA (US); James P. BISHOP, Cary, NC (US); Kenneth S. KILGORE, Berwyn, PA (US); William T. SYMONDS, Cary, NC (US); Stephen A. WRING, South Boston, VA (US); Jayne E. HASTEDT, San Carlos, CA (US); Katelyn R. CRIZER, Chapel Hill, NC (US); Michelle PALACIOS, Garner, NC (US)

(21) Appl. No.: 17/781,289

(22) PCT Filed: Dec. 2, 2020
(86) PCT No.: PCT/US20/62853

§ 371 Date: May 31, 2022

Related U.S. Application Data
(60) Provisional application No. 63/106,097, filed on Oct. 27, 2020.
(60) Provisional application No. 62/985,167, filed on Mar. 4, 2020.
(60) Provisional application No. 62/942,424, filed on Dec. 2, 2019.

(51) Int. Cl. A61K 31/5415; A61P 11/00; A61K 9/00; A61K 47/26; C07K 16/28

(52) U.S. Cl. CPC A61K 31/5415; A61P 11/00; A61K 9/0078; A61K 47/26; C07K 16/2866

(57) ABSTRACT

A method for treating an inflammatory disorder of the lower airways in a human subject in need thereof is described, including administering an effective amount of anakinra directly to the lower airways in the human subject; where the effective amount of anakinra is from about 0.1 mg to about 200 mg per day; and where the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia and pneumonitis.

This application claims the benefit of and priority under 35 U.S.C. § 119(e) to U.S. Ser. No.: 62/942,424 filed Dec. 2, 2019; U.S. Ser. No.: 62/985,167 filed Mar. 4, 2020; and U.S. Ser. No.: 63/106,097 filed Oct. 27, 2020, the contents of each of which are hereby incorporated by reference in their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### FIELD OF THE INVENTION

The invention relates generally to the field of pharmaceutical science. More particularly, the invention relates to compounds and compositions useful as pharmaceuticals for treating various lower airways disorders.

### BACKGROUND

### Bronchiolitis Obliterans Syndrome

Bronchiolitis Obliterans Syndrome (BOS) is a non-reversible lung disease characterized by obstruction of the small airways impeding airflow. BOS is one of the most common complications after lung or hematopoietic stem cell transplant and can also occur after exposure to inhaled toxins and gases. BOS patients experience shortness of breath, decreased ability to participate in daily activities, fatigue, and cough. BOS is diagnosed as a decline in lung function tests (FEV1) and confirmed with imaging (e.g., CT, X-ray). BOS disease progression involves activation of alloimmune responses and infiltration of immune cells. Additionally, the lung repair pathway is aberrantly activated causing fibroblast proliferation, activation of smooth muscle surrounding the airways, and narrowing of the lumen within the terminal and distal bronchioles. There are no currently approved FDA therapies for BOS. Other drugs that are in late-stage clinical development (e.g., inhaled cyclosporine) have a number of drawbacks, a history of failures and may not have the optimum mechanism (e.g., patients are typically already receiving a calcineurin inhibitor).

### Toxic Inhalation Lung Injury

Toxic inhalation lung injury involves damage to the lung caused by toxic agents, e.g., chemicals and irritants, carried into the lower airways. Toxic inhalation lung injury can result in varying degrees of erythema, carbonaceous deposits, bronchorrhea, severe inflammation, copious carbonaceous deposits, and/or bronchial obstruction. In the most severe cases, there is evidence of mucosal sloughing, necrosis, and endoluminal obliteration. Toxic inhalation lung injury may be associated with a broad spectrum of respiratory disorders, depending on the inhaled toxic agent.

Toxic agents with different solubility and particle sizes have differential effects on different parts of the respiratory system. Inhaled toxins with high water solubility can localize in the upper airways, while those with low water solubility tend to localize more frequently in the lower airways. Toxins of larger particle size tend to localize in the upper airways, while those of smaller particle size penetrate the lower airways.

Current treatment strategies for toxic inhalation lung injury vary depending on the specific types of toxic agents involved, the duration of exposure, and the resulting extent of injury. Treatment options may include excision of burnt tissue along with skin graft replacement, administration of normobaric oxygen, mechanical ventilation, resuscitation, and/or administration of various pharmaceutical agents. See Dries et al., J. Trauma. Resuscitation. Emerg. Med., 2013, 21, 31-45.

Inhalation of diacetyl (Kreiss, K., et al., N. Engl. J. Med., 2002, 347, 330-338; Akpinar-Elci, M., et al., European Respiratory Journal, 2004, 24, 298-302), acetaldehyde and formaldehyde, as well as other volatile compounds, has been demonstrated as a cause of bronchiolitis obliterans (BO). Inhalation exposure to these compounds in the U.S. in more recent years has occurred at commercial-scale plants, such as coffee processing facilities and popcorn production plants, where processing workers can be at risk of exposure to airborne diacetyl (2,3-butanedione), 2,3-pentanedione, and/or 2,3-hexanedione, compounds which have been linked to BO.

### Vaping-Associated Lung Injury

Vaping-associated lung injury is a newly recognized specific group of syndromes under the general category of toxic-inhalation lung injury. The existing case studies demonstrate a heterogeneous collection of pneumonitis patterns that include acute eosinophilic pneumonia, organizing pneumonia, lipoid pneumonia, diffuse alveolar damage, diffuse alveolar hemorrhage, hypersensitivity pneumonitis, and/or giant-cell interstitial pneumonitis. Regardless of the specific pneumonitis pattern, pathophysiology of vaping-associated lung injury commonly includes inflammation, edema of airways, and acute lung damage. See Butt et al., N. Engl. J. Med., 2019, 318, 1780-1781.

Several toxic compounds have been identified in vaping products: flavorants (e.g., diacetyl), nicotine, carbonyls, volatile organic compounds (e.g., benzene and toluene), trace metal elements, a-Tocopheryl acetate, and bacterial endotoxins and fungal glucans. See Christiani, D., N. Engl. J. Med., Online Editorial, Sep. 6, 2019; available at https://www.nejm.org/doi/full/10.1056/NEJMe1912032#article citing articles. There are currently no standard guidelines for treatment of vaping-associated lung injury. Patients who are admitted to hospital are typically treated with antibiotics or steroids, but many continue to exhibit abnormalities at short-term follow-up periods. See Blagev et al., The Lancet., Nov. 8, 2019; available online at https://www.thelancet.com/journals/lancet/article/PIIS0140-6736(19)32679-0/fulltext.

### Pulmonary Langerhans Cell Histiocytosis (PLCH)

Pulmonary langerhans cell histiocytosis (PLCH) is a rare disease affecting predominantly smokers. PLCH is a specific type of histiocytic syndrome characterized by accumulation of langerhans (antigen-presenting cells) and other inflammatory cells in small airways, resulting in the formation of nodular inflammatory lesions. More advanced stages are characterized by cystic lung destruction, cicatricial scarring of airways, and pulmonary vascular remodeling. PLCH often leads to death over a period of few years due to respiratory failure or malignancy. Current treatment includes corticosteroids, but it remains unclear whether this is an effective treatment option. See Murakami et al., Cell Communication and Signaling, 2015, 13, 1-15.

### Non-cystic Fibrosis Bronchiectasis

Bronchiectasis is a heterogenous chronic lung disorder characterized by recurrent cough, sputum production, and recurrent respiratory infections. Over 95% of bronchiectasis is of the non-cystic fibrosis type. Mortality rate is significant, ranging from 10 to 16% over an approximate 4-year observation period. The pathology of the disease includes dilatation of the bronchi that lead to airway inflammation and chronic bacterial colonization. Treatment typically involves a multimodal approach that includes airway clearance, anti-inflammatory agents, and inhaled antibiotics. Some patients fail to adequately respond to any currently recognized therapeutic approach and may require lobectomy or segmentectomy. See Chalmers et al., Molecular Immunology, 2013, 55, 27-34.

### Diffuse Panbronchiolitis

Diffuse panbronchiolitis is characterized by chronic sinobronchial infection, peribronchial inflammation, and significant reduction in airflow. Symptoms include crackles, wheezes, productive cough, and chronic sinusitis. Diffuse panbronchiolitis is largely resistance to bronchodilators. The first-in-line treatment involves administration of macrolides which effectively inhibit bacterial growth and reduce inflammation but can lead to several adverse side effects. See Scambler et al., Immunology, 2018, 154, 563-573.

### Acute Respiratory Distress Syndrome (ARDS)

Acute Respiratory Distress Syndrome (ARDS) is a syndrome of acute respiratory failure with progressive arterial hypoxemia, dyspnea, and/or breathlessness. The pathogenesis of ARDS involves the accumulation of protein-rich and neutrophilic pulmonary edema in the lung coupled with significant inflammation. ARDS is life threatening and requires immediate endotracheal intubation and positive pressure ventilation to prevent lung failure. There is a potential role for pharmacological treatment coupled with ventilation such as the use of glucocorticoids, surfactants, inhaled nitric oxide, antioxidants, protease inhibitors, and a variety of other anti-inflammatory agents. However, the currently available treatment options for ARDS have not been shown to be sufficiently effective. See Park et al., Am. J. Respir. Crit. Care. Med., 2001, 164, 1896-1903.

### Reactive Airways Dysfunction Syndrome (RADS)

Reactive airways dysfunction syndrome (RADS) is a persistent asthma-like disorder with sudden onset following a single acute exposure to an inhaled irritant. Chemical irritants associated with RADS may include chlorine, toluene diisocyanate (TDI), nitrogen oxides, morpholine, sulfuric acid, ammonia, and phosgene. Conventional asthma treatments can be used in RADS, including corticosteroids and bronchodilators. However, currently there are no effective treatment options for patients with persistent RADS. See Shakeri et al., Occupational Med., 2008, 58, 205-211.

### Bronchiolitis Obliterans Organizing Pneumonia (BOOP)

Bronchiolitis obliterans organizing pneumonia (BOOP) is a syndrome characterized symptomatically by subacute or chronic respiratory illness. Patients with BOOP may exhibit persistent nonproductive cough, effort dyspnea, low-grade pyrexia, and/or malaise. Pathologically, patients having BOOP may have granulation tissues in the bronchiolar lumen, alveolar ducts and alveoli, with variable degrees of inflammation. Current treatment options for BOOP are limited and typically involve oral corticosteroid therapy. A small number of patients who do not respond to standard treatment may require lung transplant. See Al-Ghanem et al., Ann. Thorac. Med., 2008, 3, 67-75.

### SUMMARY OF THE INVENTION

In one aspect, a method for treating an inflammatory disorder of the lower airways is described. In certain embodiments, the method includes administering an effective amount of anakinra directly to the lower airways in a human subject to treat inflammation of the lower airways (e.g., lung) to effectively treat the inflammatory disorder.

In one aspect, a method for treating an inflammatory disorder of lower airways in a human subject in need thereof is described, including administering an effective amount of anakinra directly to the lower airways in the human subject; where the effective amount of anakinra is from about 0.1 mg to about 200 mg per day; and where the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), and pneumonitis.

In any one of the embodiments described herein, the toxic-inhalation lung injury is caused by inhalation of one or more chemical warfare agents.

In any one of the embodiments described herein, the chemical warfare agent is selected from the group consisting of chlorine gas and sulfur mustard.

In any one of the embodiments described herein, the toxic-inhalation lung injury is caused by inhalation of one or more environmental and/or industrial toxic agents.

In any one of the embodiments described herein, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes.

In any one of the embodiments described herein, wherein the toxic-inhalation lung injury is pneumoconiosis or bronchiolitis obliterans.

In any one of the embodiments described herein, the toxic-inhalation lung injury is a vaping-associated lung injury.

In any one of the embodiments described herein, the vaping-associated lung injury is caused by inhalation of one or more agents selected from the group consisting of diacetyl, 2,3-pentanedione, α-Tocopheryl acetate, nicotine, carbonyls, benzene, toluene, metals, bacterial endotoxins, and fungal glucans.

In any one of the embodiments described herein, the inflammatory disorder is selected from the group consisting of pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), and pneumonitis. In any one of the embodiments described herein, the ARDS is associated with complications arising from viral infections caused by a virus selected from the group consisting of SARS-CoV-2, SARS-CoV, MERS-CoV, 229E, NL63, 0C43, and HKU1.

In any one of the embodiments described herein, the inflammatory disorder is an inflammatory disorder of the lung.

In any one of the embodiments described herein, anakinra is administered via inhalation or via direct instillation into the lower airways.

In any one of the embodiments described herein, anakinra is administered by a delivery device selected from the group consisting of a nebulizer, an inhaler, and a subminiature aerolizer.

In any one of the embodiments described herein, the delivery device is a mesh nebulizer.

In any one of the embodiments described herein, the delivery device is an aerosolized nebulizer.

In any one of the embodiments described herein, anakinra is administered in a pharmaceutical composition comprising anakinra and a pharmaceutically acceptable carrier.

In any one of the embodiments described herein, the pharmaceutically acceptable carrier is selected from the group consisting of saline, Ringer's solution, dextrose solution, and a combination thereof.

In any one of the embodiments described herein, the pharmaceutical composition is a spray, aerosol, gel, solution, emulsion, or suspension.

In any one of the embodiments described herein, the effective amount of anakinra is from about 0.1 mg to about 100 mg per day, from about 0.1 mg to about 50 mg per day, or from about 0.1 mg to about 10 mg per day.

In any one of the embodiments described herein, the effective amount of anakinra is from about 0.125 mg to 5.0 mg per day.

In any one of the embodiments described herein, the method further includes administering a second therapeutic agent in combination with anakinra to the human subject suffering from the inflammatory disorder of the lower airways.

In any one of the embodiments described herein, the second therapeutic agent is selected from the group consisting of an anti-inflammatory agent, an antiviral agent, an antibacterial agent, an antibiotic agent, an antifungal compound, and a mucoregulator.

In any one of the embodiments described herein, the second therapeutic agent is rodatristat ethyl.

In any one of the embodiments described herein, anakinra is administered in a pharmaceutical composition including antagonist and one or more additional components each selected from the group consisting of a buffer, a stabilizer, and a tonicity modifier.

In another aspect, a pharmaceutical composition is described, including an interleukin-1 receptor antagonist (e.g., anakinra) and one or more additional components each selected from the group consisting of a buffer, a stabilizer, and a tonicity modifier.

In any one of the embodiments described herein, the interleukin-1 receptor antagonist is anakinra.

In any one of the embodiments described herein, the buffer is selected from the group consisting of citrate, phosphate, succinate, histidine, glutamate, pyrophosphate, 4-(2-hydroxyethyl)-1-piperazineethanesulfonic acid (HEPES), and a combination thereof.

In any one of the embodiments described herein, the pharmaceutical composition is a liquid composition comprising citrate in a concertation of between about 0.5 mM and 20 mM.

In any one of the embodiments described herein, the concentration of citrate is about 20 mM.

In any one of the embodiments described herein, the pharmaceutical composition is a liquid composition comprising phosphate in a concentration of between about 1 mM and 50 mM.

In any one of the embodiments described herein, the concentration of phosphate is about 10 mM.

In any one of the embodiments described herein, the pharmaceutical composition is a liquid composition comprising histidine in a concentration of between about 5 mM and 50 mM.

In any one of the embodiments described herein, the concentration of histidine is about 10 mM.

In any one of the embodiments described herein, the pharmaceutical composition is a liquid composition comprising glutamate in a concentration of between about 1 mM and 50 mM.

In any one of the embodiments described herein, the pharmaceutical composition is a liquid composition comprising pyrophosphate in a concentration of between about 1 mM and 50 mM.

In any one of the embodiments described herein, the pharmaceutical composition is a liquid composition comprising 4-(2-hydroxyethyl)-1-piperazineethanesulfonic acid (HEPES) in a concentration of between about 10 mM and 50 mM.

In any one of the embodiments described herein, the concentration of 4-(2-hydroxyethyl)-1-piperazineethanesulfonic acid (HEPES) is about 10 mM.

In any one of the embodiments described herein, the stabilizer is selected from the group consisting of a surfactant, a chelating agent, a sugar, and a combination thereof.

In any one of the embodiments described herein, the surfactant is selected from the group consisting of polysorbate 80, polysorbate 20, polyoxyethylene (23) lauryl ether (Brij™ 35), sorbitan trioleate (Span™ 85), and a combination thereof.

In any one of the embodiments described herein, the pharmaceutical composition is a liquid composition comprising polysorbate 80 in a concentration of between about 0.01% and 1% (w/v).

In any one of the embodiments described herein, the concentration of polysorbate 80 is about 0.1% (w/v).

In any one of the embodiments described herein, the pharmaceutical composition is a liquid composition comprising polysorbate 20 in a concentration of between about 0.00001% and 1% (w/v), or between about 0.00001% and 0.01% (w/v). In any one of the embodiments described herein, the pharmaceutical composition is a liquid composition comprising polysorbate 20 in a concentration of about 0.00001% (w/v), 0.0001% (w/v), or 0.001% (w/v).

In any one of the embodiments described herein, the pharmaceutical composition is a liquid composition comprising polyoxyethylene (23) lauryl ether (Brij™ 35) in a concentration of between about 0.00001% and 0.01% (w/v).

In any one of the embodiments described herein, the pharmaceutical composition is a liquid composition comprising sorbitan trioleate (SpanTM 85) in a concentration of between about 0.1% and 5.0% (w/v), about 0.8 (w/v), 0.85 (w/v), or 0.86% (w/v).

In any one of the embodiments described herein, the chelating agent is ethylenediaminetetraacetic acid (EDTA) disodium.

In any one of the embodiments described herein, the pharmaceutical composition is a liquid composition comprising ethylenediaminetetraacetic acid (EDTA) disodium in a concentration of between about 0.05 mM and 1 mM.

In any one of the embodiments described herein, the concentration of ethylenediaminetetraacetic acid (EDTA) is about 0.5 mM.

In any one of the embodiments described herein, the sugar is selected from the group consisting of trehalose, sucrose, glycerol, sorbitol, and a combination thereof.

In any one of the embodiments described herein, the pharmaceutical composition is a liquid composition and the concentration of the sugar is greater than about 40% (w/v).

In any one of the embodiments described herein, the tonicity modifier is selected from the group consisting of sodium chloride, mannitol, taurine, hydroxyproline, proline, and a combination thereof.

In any one of the embodiments described herein, the pharmaceutical composition is a liquid composition comprising sodium chloride in a concentration of between about 120 mM and 180 mM.

In any one of the embodiments described herein, the concentration of sodium chloride is about 140 mM.

In any one of the embodiments described herein, the pharmaceutical composition is a liquid composition comprising mannitol in a concentration of between about 5 mg/mL and 50 mg/mL.

In any one of the embodiments described herein, the concentration of mannitol is about 10 mg/mL.

In any one of the embodiments described herein, the pharmaceutical composition is a liquid composition comprising taurine in a concentration of between about 15 mg/mL and 50 mg/mL.

In any one of the embodiments described herein, the concentration of taurine is about 30 mg/mL.

In any one of the embodiments described herein, the pharmaceutical composition is a liquid composition comprising hydroxyproline in a concentration of between about 15 mg/mL and 50 mg/mL.

In any one of the embodiments described herein, the concentration of hydroxyproline is about 26 mg/mL.

In any one of the embodiments described herein, the additional components comprise citrate, ethylenediaminetetraacetic acid (EDTA) disodium, polysorbate 80, and sodium chloride.

In any one of the embodiments described herein, the additional components comprise phosphate, ethylenediaminetetraacetic acid (EDTA) disodium, polysorbate 80, and sodium chloride.

In any one of the embodiments described herein, the additional components comprise phosphate, mannitol, ethylenediaminetetraacetic acid (EDTA) disodium, and sodium chloride.

In any one of the embodiments described herein, the additional components comprise phosphate, mannitol, ethylenediaminetetraacetic acid (EDTA) disodium, polysorbate 80, and sodium chloride.

In any one of the embodiments described herein, the additional components comprise phosphate, mannitol, ethylenediaminetetraacetic acid (EDTA) disodium, polysorbate 20, and sodium chloride.

In any one of the embodiments described herein, the additional components comprise phosphate, mannitol, ethylenediaminetetraacetic acid (EDTA) disodium, sorbitan trioleate (Span™ 85), and sodium chloride.

In any one of the embodiments described herein, the additional components comprise phosphate, trehalose, ethylenediaminetetraacetic acid (EDTA) disodium, polysorbate 80, and sodium chloride.

In any one of the embodiments described herein, the additional components comprise phosphate, sucrose, ethylenediaminetetraacetic acid (EDTA) disodium, polysorbate 80, and sodium chloride.

In any one of the embodiments described herein, the additional components comprise phosphate, ethylenediaminetetraacetic acid (EDTA) disodium, a tonicity modifier, and sodium chloride.

In any one of the embodiments described herein, the additional components comprise phosphate, mannitol, ethylenediaminetetraacetic acid (EDTA) disodium, polysorbate 80, a tonicity modifier, and sodium chloride.

In any one of the embodiments described herein, the additional components comprise phosphate, trehalose, ethylenediaminetetraacetic acid (EDTA) disodium, polysorbate 20, a tonicity modifier, and sodium chloride.

In any one of the embodiments described herein, the additional components comprise phosphate, sucrose, ethylenediaminetetraacetic acid (EDTA) disodium, sorbitan trioleate (Span™ 85), a tonicity modifier, and sodium chloride.

In any one of the embodiments described herein, the additional components comprise phosphate, a tonicity modifier, and sodium chloride.

In any one of the embodiments described herein, the tonicity modifier is selected from the group consisting of taurine, hydroxyproline, and a combination thereof.

In any one of the embodiments described herein, the additional components comprise citrate, phosphate, ethylenediaminetetraacetic acid (EDTA) disodium, polysorbate 80, and sodium chloride.

In any one of the embodiments described herein, the additional components comprise citrate, trehalose, ethylenediaminetetraacetic acid (EDTA) disodium, polysorbate 80, and sodium chloride.

In any one of the embodiments described herein, the additional components comprise glutamate, ethylenediaminetetraacetic acid (EDTA) disodium, polysorbate 80, and sodium chloride.

In any one of the embodiments described herein, the additional components comprise glutamate, mannitol, ethylenediaminetetraacetic acid (EDTA) disodium, polysorbate 80, and sodium chloride.

In any one of the embodiments described herein, the additional components comprise phosphate, mannitol, and sodium chloride.

In any one of the embodiments described herein, the pharmaceutical composition is a liquid composition.

In any one of the embodiments described herein, the pharmaceutical composition is a solid composition.

In any one of the embodiments described herein, the solid composition is a lyophilisate.

In any one of the embodiments described herein, the pharmaceutical composition is reconstituted from a lyophilisate.

In another aspect, a kit is disclosed, including a pharmaceutical composition according to any one of embodiments described herein and a delivery device suitable for direct administration of the pharmaceutical composition to the respiratory tract of a patient.

In any one of the embodiments described herein, the respiratory tract comprises the lower airways.

In any one of the embodiments described herein, the respiratory tract comprises the upper airways.

In any one of the embodiments described herein, the delivery device is configured to deliver an effective amount of the pharmaceutical composition via inhalation.

In any one of the embodiments described herein, the delivery device is configured to deliver an effective amount of the pharmaceutical composition via direct instillation.

In any one of the embodiments described herein, the delivery device is selected from the group consisting of a nebulizer, an inhaler, and an aerolizer.

In any one of the embodiments described herein, the delivery device is selected from the group consisting of a jet nebulizer, an ultrasonic nebulizer, a metered dose inhaler, and a dry powder inhaler.

In any one of the embodiments described herein, the nebulizer is selected from the group consisting of the Philips InnoSpire Go nebulizer and the Aerogen Solo VM nebulizer.

In any one of the embodiments described herein, the pharmaceutical composition is a liquid composition and the delivery device is configured to deliver the liquid composition.

In any one of the embodiments described herein, the pH of the liquid composition is between about 5 and 8.

In any one of the embodiments described herein, the osmolality of the liquid composition is between about 200 mOsm/kg and 400 mOsm/kg.

In any one of the embodiments described herein, the osmolality is about 300 mOsm/kg.

In any one of the embodiments described herein, the droplet size of the liquid composition produced by the delivery device is between about 0.5 μm and 10 μm in diameter.

In any one of the embodiments described herein, the droplet size of the liquid composition produced by the delivery device is suitable for preferentially targeting the lower airways.

In any one of the embodiments described herein, the droplet size of the liquid composition produced by the delivery device is between about 5 μm and 50 μm in diameter.

In any one of the embodiments described herein, the droplet size of the liquid composition produced by the delivery device is suitable for preferentially targeting the upper airways.

In any one of the embodiments described herein, the conductivity of the liquid composition is less than 2.5μS/cm.

In any one of the embodiments described herein, the pharmaceutical composition is a solid composition and the delivery device is configured to deliver the solid composition.

In any one of the embodiments described herein, the solid composition comprises particles having a mass median aerodynamic diameter (MMAD) between about 0.1 μm and 20 μm.

In any one of the embodiments described herein, the MMAD of the particles is less than about 5 μm.

In any one of the embodiments described herein, the MMAD of the particles is less than about 3.5 μm.

In any one of the embodiments described herein, the solid composition comprises particles having a mass median diameter (MMD) between about 0.1 μm and 20 μm.

In any one of the embodiments described herein, the solid composition comprises particles having a mass median aerodynamic diameter (MMAD) between about 1 μm and 5 μm and a mass median diameter (MMD) between about 5 μm and 30 μm.

In any one of the embodiments described herein, the ratio of MMD to MMAD is between about 2 and 30.

In any one of the embodiments described herein, the ratio of MMD to MMAD is between about 5 and 30.

In any one of the embodiments described herein, the solid composition has a tap density of less than about 1 g/cm³.

In any one of the embodiments described herein, the solid composition has a rugosity between about 1 and 6.

In any one of the embodiments described herein, the solid composition comprises porous particles.

In any one of the embodiments described herein, the solid composition comprises swellable particles.

In any one of the embodiments described herein, the porous particles comprise biodegradable polymers.

In any one of the embodiments described herein, the solid composition further comprises a salt of a fatty acid or a derivative thereof.

In any one of the embodiments described herein, the salt is selected from the group consisting of magnesium stearate, sodium stearyl fumarate, sodium stearyl lactylate, sodium lauryl sulfate, magnesium lauryl sulfate, and a combination thereof.

In any one of the embodiments described herein, the solid composition comprises particles having uniform particle size distribution.

In any one of the embodiments described herein, the solid composition comprises particles having nonuniform particle size distribution.

In any one of the embodiments described herein, the solid composition comprises particles having bimodal particle size distribution.

In any one of the embodiments described herein, the percent mass of the interleukin-1 antagonist in the solid composition is between about 1% and 40%, 40% and 70%, or more than 70%.

In any one of the embodiments described herein, the solid composition comprises a plurality of particles enclosed in a plurality of receptacles.

In any one of the embodiments described herein, the receptacles are selected from the group consisting of capsules, blisters, and film covered containers.

In any one of the embodiments described herein, the delivery device is suitable for direct administration of the pharmaceutical composition to bronchioles.

In any one of the embodiments described herein, the delivery device is suitable for direct administration of the pharmaceutical composition to alveolar tissue.

In yet another aspect, a method of treating an inflammatory disorder of the respiratory tract is disclosed, including administering to a patient in need thereof the pharmaceutical composition according to any one of the embodiments described herein.

In any one of the embodiments described herein, the inflammatory disorder of the respiratory tract is an inflammatory disorder of the upper airways.

In any one of the embodiments described herein, the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), bronchiolitis obliterans syndrome (BOS), idiopathic pulmonary fibrosis (IPF), pneumonitis, primary graft dysfunction (PGD), and reperfusion injury.

In any one of the embodiments described herein, the toxic-inhalation lung injury is caused by inhalation of one or more chemical warfare agents.

In any one of the embodiments described herein, the chemical warfare agent is selected from the group consisting of chlorine gas and sulfur mustard.

In any one of the embodiments described herein, the toxic-inhalation lung injury is chlorine-induced bronchiolitis obliterans syndrome (BOS) and sulfur mustard-induced bronchiolitis obliterans syndrome (BOS).

In any one of the embodiments described herein, the toxic-inhalation lung injury is caused by inhalation of one or more environmental and/or industrial toxic agents.

In any one of the embodiments described herein, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes.

In any one of the embodiments described herein, the toxic-inhalation lung injury is pneumoconiosis or bronchiolitis obliterans.

In any one of the embodiments described herein, the toxic-inhalation lung injury is a vaping-associated lung injury.

In any one of the embodiments described herein, the vaping-associated lung injury is caused by inhalation of one or more agents selected from the group consisting of diacetyl, a-Tocopheryl acetate, 2,3-pentanedione, nicotine, carbonyls, benzene, toluene, metals, bacterial endotoxins, and fungal glucans.

In any one of the embodiments described herein, the inflammatory disorder is an inflammatory disorder of the lung.

In any one of the embodiments described herein, the inflammatory disorder of the respiratory tract is an inflammatory disorder of the lower airways.

In any one of the embodiments described herein, a sustained exposure of the pharmaceutical composition in a lung epithelial lining fluid is between about 15 hours and about 100 hours. In any one of the embodiments described herein, the sustained exposure of the pharmaceutical composition in the lung epithelial lining fluid is at least 24 hours.

In any one of the embodiments described herein, the pharmaceutical composition is administered between about once per week and about three times per day. In any one of the embodiments described herein, the pharmaceutical composition is administered about once or twice daily.

In any one of the embodiments described herein, the pharmaceutical composition is administered via inhalation for between about 3 minutes and about 20 minutes.

In any one of the embodiments described herein, the pharmaceutical composition is administered at a dose of between about 0.5 mg/kg and about 2 mg/kg.

In any one of the embodiments described herein, the pharmaceutical composition binds with substantially similar affinity as an endogenous IL-1β ligand to an IL-1 type 1 receptor.

### BRIEF DESCRIPTION OF THE DRAWINGS

The following figures depict illustrative embodiments of the invention.

FIG. 1 illustrates a mechanism of action of an rhIL-1Ra that targets both innate and adaptive immune responses mediated by IL-1 signaling.

FIG. 2 shows a checkerboard table for various embodiments of ALTA-2530 formulations.

FIG. 3 illustrates a tiered approach for preformulation studies of ALTA-2530.

FIG. 4 illustrates a phase 1 study protocol using single and/or multiple ascending doses (SAD/MAD) of ALTA-2530 in healthy volunteers and BOS patients.

FIG. 5 illustrates a phase 2b/3 study of ALTA-2530 in BOS patients with 12 week POC interim.

FIG. 6 is a plot showing concentration versus time profiles for rhIL-1Ra in ELF and serum following single doses to rat.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The term “upper airways” or “upper respiratory tract” when used herein refers to or describes the anatomic regions including the passageways from flares or nostrils to the soft palate and includes the sinuses.

The term “lower airways” or “lower respiratory tract” when used herein refers to or describes the anatomic regions below the larynx including the trachea and lungs.

The terms “treating,” “treatment,” and “therapy” as used herein refer to attempted reduction or amelioration of the progression, severity and/or duration of a disorder, or the attempted amelioration of one or more symptoms thereof resulting from the administration of one or more modalities (e.g., one or more therapeutic agents such as a compound or composition of the invention).

As used herein, the terms “prevent,” “preventing” and “prevention” refer to the prevention or inhibiting of the recurrence, onset, or development of a disorder or a symptom thereof in a subject resulting from the administration of a therapy (e.g., a prophylactic or therapeutic agent), or the administration of a combination of therapies (e.g., a combination of prophylactic or therapeutic agents).

As used herein, “therapeutically effective amount” or “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the terms “subject” and “patient” are used interchangeably herein. The terms “subject” and “subjects” refer to an animal, preferably a mammal including a nonprimate and a primate (e.g., a monkey such as a cynomolgus monkey, a chimpanzee, and a human), and more preferably a human. The term “animal” also includes, but is not limited to, companion animals such as cats and dogs; zoo animals; wild animals; farm or sport animals such as ruminants, non-ruminants, livestock and fowl (e.g., horses, cattle, sheep, pigs, turkeys, ducks, and chickens); and laboratory animals, such as rodents (e.g., mice, rats), rabbits; and guinea pigs, as well as animals that are cloned or modified, either genetically or otherwise (e.g., transgenic animals).

As used herein, “a” or “an” means at least one, unless clearly indicated otherwise. The term “about,” unless otherwise indicated, refers to a value that is no more than 10% above or below the value being modified by the term. For example, the term “about 5% (w/w)” means a range of from 4.5% (w/w) to 5.5% (w/w).

As used herein, unless indicated otherwise, the terms “composition” and “composition of the invention”, are used interchangeably. Unless stated otherwise, the terms are meant to encompass, and are not limited to, pharmaceutical compositions and nutraceutical compositions containing drug substance (e.g., anakinra). The composition may also contain one or more “excipients” that are inactive ingredients or compounds devoid of pharmacological activity or other direct effect in the diagnosis, cure, mitigation, treatment, or prevention of disease or to affect the structure or any function of the human.

As used herein, the term “vehicle” refers to a diluent, adjuvant, excipient, carrier, or filler with which the compound or composition of the invention is stored, transported, and/or administered.

As used herein, the phrase “pharmaceutically acceptable salt” refers to pharmaceutically acceptable organic or inorganic salts of a compound of the invention.

As used herein, the term “pharmaceutically acceptable solvate” refers to an association of one or more solvent molecules and a compound of the invention. Examples of solvents that form pharmaceutically acceptable solvates include, but are not limited to water, saline, water-salt mixtures, isopropanol, ethanol, methanol, DMSO, ethyl acetate, acetic acid, polyethylene glycol and ethanolamine.

As used herein, the term “pharmaceutically acceptable” means approved by a regulatory agency of the Federal or a state government or listed in the U.S. Pharmacopeia or other generally recognized pharmacopeia for use in animals, and more particularly in humans.

### Method of Treating Inflammatory Disorders

In one aspect, a method for treating an inflammatory disorder is described. In certain embodiments, the method includes administering an effective amount of anakinra directly to the lower airways in the human subject. Anakinra is a recombinant and modified version of the human interleukin-1 receptor antagonist protein (rhIL-1ra). In some embodiments, the the method includes administering an effective amount of ALTA-2530, which is a novel formulation of anakinra as described herein. In some embodiments, ALTA-2530 is an inhaled formulation of anakinra.

IL-1 receptor activity induces a myriad of secondary inflammatory mediators, including prostaglandins, cytokines, and chemokines. Anakinra blocks the biologic activity of the endogenous IL-1α and IL-1β cytokines, which are part of the Interleukin-1 family (“IL-1”), by competitively inhibiting IL-la and IL-1(3′s binding to the interleukin-1 type I receptor (IL-1RI).

It has been surprisingly found that anakinra can be directly administered to the lower airways, e.g., the lung, in the human subject having the inflammatory disorder disclosed herein to effectively treat the inflammatory disorder. In some embodiments, the inflammatory disorder disclosed herein is associated with or at least in part associated with the lower airways. Without wishing to be bound by any specific theory, it is believed that in the human subject having the inflammatory disorder disclosed herein, IL-1α and IL-1β bind to the interleukin-1 type I receptor which triggers inflammation and that anakinra blocks the activities of these IL-1 cytokines local to the lower airways, thereby effectively treating inflammation and the inflammatory disorder.

Thus, in one aspect, a method for treating an inflammatory disorder of lower airways in a human subject in need thereof is described, including administering an effective amount of anakinra directly to the lower airways in the human subject; and where the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), and pneumonitis. In some embodiments, the inflammatory disorder is an inflammatory disorder of the lung. In some embodiments, the inflammatory disorder is pneumonitis or pneumoconiosis. In some embodiments, the effective amount of anakinra is from about 0.1 mg to about 200 mg per day.

As used herein, a toxic-inhalation lung injury includes any injury (e.g., inflammation or damage) to the lung as a result of inhalation of one or more foreign and/or toxic agents. In some embodiments, the subject having a toxic-inhalation lung injury suffers from inflammation mediated by IL-1α and IL-1β and anakinra blocks the activities of these cytokines local to the lower airways and thereby effectively treats inflammation and the toxic-inhalation lung injury.

In some embodiments, the toxic-inhalation lung injury is caused by inhalation of one or more chemical warfare agents. Non-limiting examples of the chemical warfare agents include chlorine gas and sulfur mustard. There is no approved treatment for sulfur mustard inhalation injury. Mustard gas causes acute effects such as bleeding/blistering in the lungs, damage to mucous membranes, and pulmonary edema, and chronic effects such as parenchymal fibrosis and BOS. Other examples of the chemical warfare agents known in the art are contemplated. Chlorine gas causes acute effects such as acute pulmonary edema, airway hyperresponsiveness, and inflammasome activation, and chronic effects such as airway hyperresponsiveness and BOS. In some embodiments, the toxic-inhalation lung injury is chlorine-induced BOS. In some embodiments, the toxic-inhalation lung injury is sulfur mustard-induced BOS.

In other embodiments, the toxic-inhalation lung injury is caused by inhalation of one or more environmental and/or industrial toxic agents. Various toxic agents exist in the environmental (natural or artificial) and industrial setting. A human subject may come into contact with and inhale these agents (e.g., while working) and suffer from injuries to the lung that lead to inflammation. Non-limiting examples of the environmental and industrial toxic agents include isocyanate (e.g., toluene diisocyanate), nitrogen oxide, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes (e.g., fumes generated by copper, magnesium, nickel, silver, or zinc). In some specific embodiments, the toxic-inhalation lung injury is pneumoconiosis. As used herein, pneumoconiosis refers to a class of interstitial lung diseases caused by inhalation of various solid particles. In some specific embodiments, the toxic-inhalation lung injury is bronchiolitis obliterans, commonly referred to as “popcorn lung.” In some specific embodiments, the bronchiolitis obliterans is caused by the inhalation of one or more industrial toxic agents selected from the group consisting of acetaldehyde, formaldehyde, diacetyl, 2,3-pentanedione, and 2,3-hexanedione.

In still other embodiments, the toxic-inhalation lung injury is a vaping-associated lung injury. Vaping, or using electronic cigarettes, may cause the user to inhale harmful chemicals and result in lung injuries. In recent years, there has been a significant growth in cases of vaping-associated lung injury reported in the U.S. In some embodiments, an electronic cigarette user inhales harmful chemicals found in the electronic cigarette liquid, e.g., diacetyl, which causes injuries, e.g., burn, inflammation, to the lung. Other non-limiting examples of harmful chemicals in the electronic cigarette causing vaping-associated lung injury include 2,3-pentanedione, nicotine, carbonyls, volatile organic compounds (e.g., benzene and toluene), trace metal elements, α-Tocopheryl acetate, and bacterial endotoxins and fungal glucans. In some embodiments, the vaping-associated lung injury is pneumonitis. In some specific embodiments, the vaping-associated lung injury is bronchiolitis obliterans, commonly referred to as “popcorn lung.”

In yet other embodiments, the inflammatory disorder is selected from the group consisting of pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), and pneumonitis.

Pulmonary langerhans cell histiocytosis is a lung disease more commonly occurring in smokers. In some embodiments, the subject having pulmonary langerhans cell histiocytosis suffers from inflammation mediated by IL-1 and anakinra blocks the activities of IL-1α and IL-1β local to the lower airways and thereby effectively treat pulmonary langerhans cell histiocytosis.

In non-cystic fibrosis bronchiectasis and in diffuse panbronchiolitis disease, various biological pathways are activated which induce inflammation of the lung. In some embodiments, the subject having non-cystic fibrosis bronchiectasis of the lower airways suffers from inflammation mediated by IL-1 and anakinra blocks the activities of IL-1α, IL-1β local to the lower airways and thereby effectively treats non-cystic fibrosis bronchiectasis. In other embodiments, the subject having diffuse panbronchiolitis of the lower airways suffers from inflammation mediated by IL-1 and anakinra blocks the activities of IL-1α, IL-1β local to the lower airways and thereby effectively treats diffuse panbronchiolitis.

Acute respiratory distress syndrome (ARDS) is a life-threatening disease which requires immediate mechanical ventilation to prevent lung failure. In some embodiments, ARDS is associated with complications arising from viral infections caused by SARS-CoV-2, SARS-CoV, MERS-CoV, 229E, NL63, 0C43, and HKU1. In some embodiments, the subject having ARDS suffers from inflammation mediated by IL-1 and anakinra blocks the activities of IL-1α, IL-1β local to the lower airways and thereby effectively treats ARDS.

In some embodiments, the human subject to be treated suffers from reactive airways dysfunction syndrome (RADS), which refers to a persistent asthma-like disorder precipitated by a single acute exposure to an inhaled irritant. In some embodiments, the subject having RADS suffers from inflammation mediated by IL-1 and anakinra blocks the activities of IL-1α, IL-1β local to the lower airways and thereby effectively treats RADS.

In some embodiments, the human subject to be treated suffers from bronchiolitis obliterans organizing pneumonia (BOOP), which is a type of lung disease resulting from organizing pneumonia that invades the bronchioles (small airways through the lungs) and alveoli (tiny air sacs) of the lungs. BOOP causes inflammation of the bronchioles and alveoli of the lung. In some embodiments, the subject having BOOP suffers from inflammation mediated by IL-1 and anakinra blocks the activities of IL-1α, IL-1β local to the lower airways and thereby effectively treats BOOP.

In some embodiments, the human subject to be treated suffers from BOS. In BOS, host T cells recognize foreign antigens and infiltrate the lung to induce acute rejection. BOS occurs following chronic rejection and infiltration of T cells, B cells, and innate immune cells. Immune cells promote fibrosis and airway occlusion. Injury activates DAMPs/PAMPs which induce inflammasome activation and IL-1 release. IL-1 drives the innate immune response which increases inflammatory cytokine production by macrophages, dendritic cells, and mast cells. IL-1 also increases neutrophil recruitment and effector function, and stimulates release of IFN-y from NK cells. IL-1 also stimulates adaptive immunity including promoting the generation of CD8+T cells and release of cytotoxic granzyme B. The role of IL-1 in adaptive immunity also enhances CD4+T cell populations and differentiation into Th17 helper T cells, aids in memory T cell functions and priming of T cells, and promotes cytotoxic cytokine release.

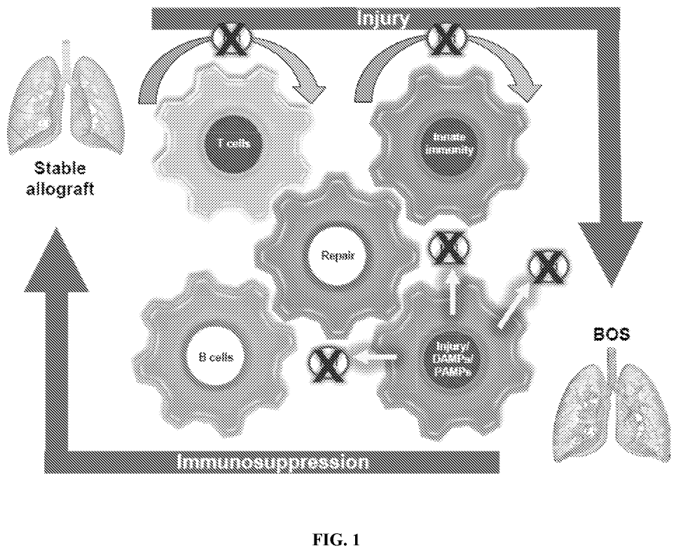

In some embodiments, a human subject suffering from BOS is administered a rhIL-1Ra, such as anakinra, to the lower airways. In some embodiments, the formulation of anakinra is ALTA-2530. In some embodiments, the rhIL-1Ra inhibits IL-1 signaling, blocking both IL-α and IL-1β signaling. FIG. 1 depicts a mechanism of action of an rhIL-1Ra, such as ALTA-2530, which both innate and adaptive immune reactions mediated by IL-1 signaling (both IL-α and IL-1β signaling).

In some embodiments, anakinra is administered via inhalation or via direct instillation into the lower airways. In certain embodiments, a delivery device is used to administer anakinra directly to the lower airways. Non-limiting examples of the delivery devices include a nebulizer, an inhaler, and a subminiature aerolizer. In some specific embodiments, the delivery device is a dry powder inhaler. In some specific embodiments, the delivery device is a mesh nebulizer.

### Second Therapeutic Agent

In some embodiments, the method described herein further includes administering a second therapeutic agent in combination with anakinra to the human subject suffering from an inflammatory disorder of the lower airways. In some embodiments, the second therapeutic agent is selected from the group consisting of an anti-inflammatory agent, an antiviral agent, an antibacterial agent, an antibiotic, an antifungal compound, an amiloride, an antihistamine, an anticholinergic, a mucolytic, and a steroid. In some specific embodiments, the second therapeutic agent is rodatristat ethyl.

In one embodiment, a proinflammatory cytokine inhibitor (e.g., anakinra) may also be administered along with other active or pharmacologic agents, such as UTP, amiloride, antibiotics, antihistamines, anti-cholinergics, anti-inflammatory agents, and mucolytics (e.g., n-acetyl-cysteine). It may also be useful to administer the proinflammatory cytokine inhibitor (e.g., anakinra) along with other therapeutic human proteins including but not limited to serine and other protease inhibitors, gamma-interferon, enkephalinase, nucleases, colony stimulating factors, albumin, and antibodies. The proinflammatory cytokine inhibitor may be administered sequentially or concurrently with the one or more other pharmacologic agents. The amounts of proinflammatory cytokine inhibitor (e.g., anakinra) and pharmacologic agent depend, for example, on what type of drugs are used, the type of lower airways inflammatory disorder being treated, and the scheduling and routes of administration. Following administration of proinflammatory cytokine inhibitor to the mammal (e.g., a human), the mammal's physiological condition can be monitored in various ways well known to one of ordinary skill in the art.

In another embodiment, the composition used for treating disorders of the lower airways may comprise a proinflammatory cytokine inhibitor (e.g., anakinra or ALTA-2530) and other compounds including but not limited to a mucoregulatory compound, a corticosteroid, a surfactant, an anticholinergic compound, a bronchodilator, a nuclease, an antibiotic, an antiviral agent, and an antiangiogenic agent. Additional examples of the second therapeutic agents are disclosed in U.S. Pat. No. 8,940,683, cols. 23-32 and 47-51, the content of which is expressly incorporated herein by reference.

### Compositions of Interleukin-1 Receptor Antagonists

In certain embodiments, a novel pharmaceutical composition comprising an ILl-ra is described, referred to herein as ALTA-2530. In some embodiments, ALTA-2530 is delivered directly to target tissue via inhalation which solves for impaired perfusion in post-LT patients and limits systemic side effects. In some embodiments, ALTA-2530 has a novel mechanism of action which targets innate immune response in BOS. In some embodiments, ALTA-2530 exhibits a strong safety profile based on successful early clinical experience.

In certain embodiments, a pharmaceutical composition is described, including an interleukin-1 receptor antagonist and one or more additional components each selected from the group consisting of a buffer, a stabilizer, and a tonicity modifier.

In some embodiments, the interleukin-1 receptor antagonist is anakinra. Other interleukin-1 receptor antagonists are contemplated. In certain embodiments, the pharmaceutical compositions described herein are examples for formulations of anakinra for nebulized delivery.

In some embodiments, the buffer is selected from the group consisting of citrate, phosphate, succinate, histidine, glutamate, pyrophosphate, 4-(2-hydroxyethyl)-1-piperazineethanesulfonic acid (HEPES), and a combination thereof. In some embodiments, the pharmaceutical composition is a liquid composition comprising citrate in a concertation of between about 0.5 mM and 20 mM.

In some embodiments, the concentration of citrate is about 20 mM. In some embodiments, the pharmaceutical composition is a liquid composition comprising phosphate in a concentration of between about 1 mM and 50 mM, or about 10 mM.

In some embodiments, the pharmaceutical composition is a liquid composition comprising histidine in a concentration of between about 5 mM and 50 mM or about 10 mM.

In some embodiments, the pharmaceutical composition is a liquid composition comprising glutamate in a concentration of between about 1 mM and 50 mM. In some embodiments, the pharmaceutical composition is a liquid composition comprising pyrophosphate in a concentration of between about 1 mM and 50 mM.

In some embodiments, the pharmaceutical composition is a liquid composition comprising 4-(2-hydroxyethyl)-1-piperazineethanesulfonic acid (HEPES) in a concentration of between about 10 mM and 50 mM or about 10 mM.

In some embodiments, the stabilizer is selected from the group consisting of a surfactant, a chelating agent, a sugar, and a combination thereof In some embodiments, the surfactant is selected from the group consisting of polysorbate 80, polysorbate 20, polyoxyethylene (23) lauryl ether (Brij™ 35), sorbitan trioleate (Span™ 85), and a combination thereof.

In some embodiments, the pharmaceutical composition is a liquid composition comprising polysorbate 80 in a concentration of between about 0.01% and 1% (w/v) or about 0.1% (w/v).

In some embodiments, the pharmaceutical composition is a liquid composition comprising polysorbate 20 in a concentration of between about 0.00001% and 1% (w/v), or between about 0.00001% and 0.01% (w/v). In any one of the embodiments described herein, the pharmaceutical composition is a liquid composition comprising polysorbate 20 in a concentration of about 0.00001% (w/v), 0.0001% (w/v), or 0.001% (w/v).

In some embodiments, the pharmaceutical composition is a liquid composition comprising polyoxyethylene (23) lauryl ether (Brij™ 35) in a concentration of between about 0.00001% and 0.01% (w/v). In some embodiments, the pharmaceutical composition is a liquid composition comprising sorbitan trioleate (Span™ 85) in a concentration of between about 0.1% and 5.0% (w/v), about 0.8 (w/v), 0.85 (w/v), or 0.86% (w/v).

In some embodiments, the chelating agent is ethylenediaminetetraacetic acid (EDTA) disodium. In some embodiments, the pharmaceutical composition is a liquid composition comprising ethylenediaminetetraacetic acid (EDTA) disodium in a concentration of between about 0.05 mM and 1 mM or about 0.5 mM.

In some embodiments, the sugar is selected from the group consisting of trehalose, sucrose, glycerol, sorbitol, and a combination thereof. In some embodiments, the pharmaceutical composition is a liquid composition and the concentration of the sugar is greater than about 40% (w/v).

In some embodiments, the tonicity modifier is selected from the group consisting of sodium chloride, mannitol, taurine, hydroxyproline, proline, and a combination thereof In some embodiments, the pharmaceutical composition is a liquid composition comprising sodium chloride in a concentration of between about 120 mM and 180 mM or about 140 mM.

In some embodiments, the pharmaceutical composition is a liquid composition comprising mannitol in a concentration of between about 5 mg/mL and 50 mg/mL or about 10 mg/mL.

In some embodiments, the pharmaceutical composition is a liquid composition comprising taurine in a concentration of between about 15 mg/mL and 50 mg/mL or about 30 mg/mL.

In some embodiments, the pharmaceutical composition is a liquid composition comprising hydroxyproline in a concentration of between about 15 mg/mL and 50 mg/mL or about 26 mg/mL.

In some embodiments, the additional components comprise citrate, ethylenediaminetetraacetic acid (EDTA) disodium, polysorbate 80, and sodium chloride. In some embodiments, the additional components comprise phosphate, ethylenediaminetetraacetic acid (EDTA) disodium, polysorbate 80, and sodium chloride.

In some embodiments, the additional components comprise phosphate, mannitol, ethylenediaminetetraacetic acid (EDTA) disodium, and sodium chloride. In some embodiments, the additional components comprise phosphate, mannitol, ethylenediaminetetraacetic acid (EDTA) disodium, polysorbate 80, and sodium chloride.

In some embodiments, the additional components comprise phosphate, mannitol, ethylenediaminetetraacetic acid (EDTA) disodium, polysorbate 20, and sodium chloride. In some embodiments, the additional components comprise phosphate, mannitol, ethylenediaminetetraacetic acid (EDTA) disodium, sorbitan trioleate (Span™ 85), and sodium chloride.

In some embodiments, the additional components comprise phosphate, trehalose, ethylenediaminetetraacetic acid (EDTA) disodium, polysorbate 80, and sodium chloride. In some embodiments, the additional components comprise phosphate, sucrose, ethylenediaminetetraacetic acid (EDTA) disodium, polysorbate 80, and sodium chloride.

In some embodiments, the additional components comprise phosphate, ethylenediaminetetraacetic acid (EDTA) disodium, a tonicity modifier, and sodium chloride. In some embodiments, the additional components comprise phosphate, mannitol, ethylenediaminetetraacetic acid (EDTA) disodium, polysorbate 80, a tonicity modifier, and sodium chloride.

In some embodiments, the additional components comprise phosphate, trehalose, ethylenediaminetetraacetic acid (EDTA) disodium, polysorbate 20, a tonicity modifier, and sodium chloride. In some embodiments, the additional components comprise phosphate, sucrose, ethylenediaminetetraacetic acid (EDTA) disodium, sorbitan trioleate (Span™ 85), a tonicity modifier, and sodium chloride.

In some embodiments, the additional components comprise phosphate, a tonicity modifier, and sodium chloride. In some embodiments, the tonicity modifier is selected from the group consisting of taurine, hydroxyproline, and a combination thereof.

In some embodiments, the additional components comprise citrate, phosphate, ethylenediaminetetraacetic acid (EDTA) disodium, polysorbate 80, and sodium chloride. In some embodiments, the additional components comprise glutamate, ethylenediaminetetraacetic acid (EDTA) disodium, polysorbate 80, and sodium chloride.

In some embodiments, the additional components comprise citrate, trehalose, ethylenediaminetetraacetic acid (EDTA) disodium, polysorbate 80, and sodium chloride.

In some embodiments, the additional components comprise glutamate, mannitol, ethylenediaminetetraacetic acid (EDTA) disodium, polysorbate 80, and sodium chloride. In some embodiments, the additional components comprise phosphate, mannitol, and sodium chloride.

In some embodiments, the pharmaceutical composition is a liquid composition. In some embodiments, the pharmaceutical composition is a solid composition.

In some embodiments, the solid composition is a lyophilisate. In some embodiments, the pharmaceutical composition is reconstituted from a lyophilisate.

In another aspect, a kit is disclosed, including a pharmaceutical composition according to any one of embodiments described herein and a delivery device suitable for direct administration of the pharmaceutical composition to the respiratory tract of a patient.

In some embodiments, the respiratory tract comprises the lower or upper airways.

In some embodiments, the delivery device is configured to deliver an effective amount of the pharmaceutical composition via inhalation. In some embodiments, the delivery device is configured to deliver an effective amount of the pharmaceutical composition via direct instillation.

In some embodiments, the delivery device is selected from the group consisting of a nebulizer, an inhaler, and an aerolizer. In some embodiments, the delivery device is selected from the group consisting of a jet nebulizer, an ultrasonic nebulizer, a metered dose inhaler, and a dry powder inhaler. In some embodiments, the nebulizer is selected from the group consisting of the Philips InnoSpire Go nebulizer, the AeroEclipse II jet nebulizer, and the Aerogen Solo VM nebulizer.

In some embodiments, the pharmaceutical composition is a solution for nebulization delivered using a Philips InnoSpire Go vibrating mesh (VM) nebulizer. In some embodiments, the pharmaceutical composition is a solution for nebulization that will be delivered using a preclinical nebulizer (Aerogen Solo VM nebulizer). In some embodiments, the pharmaceutical composition is an extemporaneously prepared solution formulation for nebulization that can be produced at the preclinical and clinical study sites and is stable for nebulization over the dosing period and a minimum in-use period of 24 hours. In some embodiments, the pharmaceutical composition is a solution for nebulization stored at refrigeration temperatures. In some embodiments, the pharmaceutical composition developed for GLP toxicology and GMP clinical studies will preferably be the same or comparable to avoid any bridging studies (e.g., excipients will not differ, and ratios will not exceed GLP qualification levels). In some embodiments, the pharmaceutical composition's impurity profiles of the nebulized GMP clinical formulation will be similar to and will not exceed the impurity limits qualified in the GLP preclinical studies. In some embodiments, the pharmaceutical composition is a clinical formulation solution having concentration(s) suited to deliver 10-40 mg from the VM nebulizer (expressed as drug charge to nebulizer) in less than 5 minutes and ideally within 2-3 minutes using the Philips InnoSpire Go nebulizer. In some embodiments, the pharmaceutical composition is reproducibly delivered, and pulmonary lung dose supports the clinical programs as demonstrated by chemical and aerosol performance stability over the in-use period and anticipated dosing duration. In some embodiments, the pharmaceutical composition has tolerability similar to or greater than thresholds qualified in preclinical freeze/thaw studies. In some embodiments, the pharmaceutical composition is stable based on preclinical stress stability studies. In some embodiments, the pharmaceutical composition meets purity standards based on preclinical filter compatibility studies. In some embodiments, the pharmaceutical composition does not exceed loss of content thresholds based on filter compatibility preclinical studies. In some embodiments, the pharmaceutical composition's stability of the nebulized GMP clinical formulation is similar to or greater than the stability thresholds qualified in preclinical studies. In some embodiments, the pharmaceutical composition's in-use period of the nebulized GMP clinical formulation is similar to the in-use period qualified in preclinical studies. In some embodiments, the pharmaceutical composition's storage conditions of the nebulized GMP clinical formulation is similar to the storage conditions qualified in preclinical studies. In some embodiments, the pharmaceutical composition's pH, osmolality, and appearance are similar to measures qualified in preclinical studies. In some embodiments, a protein concentration of the pharmaceutical composition is similar to a concentration qualified in preclinical studies. In some embodiments, purity of the pharmaceutical composition is similar to measures qualified in RPHPLC, SE-HPLC, reduced and non-reduced CE-SDS, and IEX-HPLC preclinical studies. In some embodiments, the levels of foreign and particulate matter, and subvisible particles in the pharmaceutical composition are similar to levels qualified in preclinical studies. In some embodiments, the levels of foreign and particulate matter, and subvisible particles in the pharmaceutical composition are similar to levels qualified in preclinical studies. In some embodiments, the pharmaceutical composition's aerosol particle size distribution by NGI of the nebulized GMP clinical formulation will be similar to the particle size distribution listed in USP 601. In some embodiments, the pharmaceutical composition's delivered dose using breath simulator will be similar to the dose listed in USP 1601 and USP 601 over the entire duration of dosing. In some embodiments, the pharmaceutical composition's potency will be similar to potency qualified in preclinical cell-based bioassay studies. In some embodiments, the pharmaceutical composition's measure of circular dichroism, viscosity, surface tension, formulation density, droplet size and distribution (e.g., as measured by Malvern Spraytec or equivalent), dynamic light scattering (DLS), and turbidity will be similar to measures qualified in preclinical studies.

In some embodiments, the pharmaceutical composition is a liquid composition and the delivery device is configured to deliver the liquid composition. In some embodiments, the pH of the liquid composition is between about 5 and 8.

In some embodiments, the osmolality of the liquid composition is between about 200 mOsm/kg and 400 mOsm/kg. In some embodiments, the osmolality is about 300 mOsm/kg.

In some embodiments, the droplet size of the liquid composition produced by the delivery device is between about 0.5 μm and 10 μm in diameter. In some embodiments, the droplet size of the liquid composition produced by the delivery device is suitable for preferentially targeting the lower airways.

In some embodiments, the droplet size of the liquid composition produced by the delivery device is between about 5 μm and 50 μm in diameter. In some embodiments, the droplet size of the liquid composition produced by the delivery device is suitable for preferentially targeting the upper airways. In some embodiments, the conductivity of the liquid composition is less than 2.50 μS/cm.

In some embodiments, the pharmaceutical composition is a solid composition and the delivery device is configured to deliver the solid composition. In some embodiments, the solid composition comprises particles having a mass median aerodynamic diameter (MMAD) between about 0.1 μm and 20 μm. In some embodiments, the MMAD of the particles is less than about 5 μm. In some embodiments, the MMAD of the particles is less than about 3.5 μm.

In some embodiments, the solid composition comprises particles having a mass median diameter (MMD) between about 0.1 μm and 20 μm. In some embodiments, the solid composition comprises particles having a mass median aerodynamic diameter (MMAD) between about 1 μm and 5 μm and a mass median diameter (MMD) between about 5 μm and 30 μm. In some embodiments, the ratio of MMD to MMAD is between about 2 and 30. In some embodiments, the ratio of MMD to MMAD is between about 5 and 30.

In some embodiments, the solid composition has a tap density of less than about 1 g/cm³. In some embodiments, the solid composition has a rugosity between about 1 and 6.

In some embodiments, the solid composition comprises porous particles. In some embodiments, the solid composition comprises swellable particles.

In some embodiments, the porous particles comprise biodegradable polymers. In some embodiments, the solid composition further comprises a salt of a fatty acid or a derivative thereof.

In some embodiments, the salt is selected from the group consisting of magnesium stearate, sodium stearyl fumarate, sodium stearyl lactylate, sodium lauryl sulfate, magnesium lauryl sulfate, and a combination thereof. In some embodiments, the solid composition comprises particles having uniform particle size distribution.

In some embodiments, the solid composition comprises particles having nonuniform particle size distribution. In some embodiments, the solid composition comprises particles having bimodal particle size distribution.

In some embodiments, the percent mass of the interleukin-1 antagonist in the solid composition is between about 1% and 40%, 40% and 70%, or more than 70%.

In some embodiments, the solid composition comprises a plurality of particles enclosed in a plurality of receptacles. In some embodiments, the receptacles are selected from the group consisting of capsules, blisters, and film covered containers. In some embodiments, the delivery device is suitable for direct administration of the pharmaceutical composition to bronchioles. In some embodiments, the delivery device is suitable for direct administration of the pharmaceutical composition to alveolar tissue.

In yet another aspect, a method of treating an inflammatory disorder of the respiratory tract is disclosed, including administering to a patient in need thereof the pharmaceutical composition according to any one of the embodiments described herein.

In some embodiments, the inflammatory disorder of the respiratory tract is an inflammatory disorder of the upper airways. In some embodiments, the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), bronchiolitis obliterans syndrome (BOS), idiopathic pulmonary fibrosis (IPF), pneumonitis, primary graft dysfunction (PGD), and reperfusion injury.

In some embodiments, the toxic-inhalation lung injury is caused by inhalation of one or more chemical warfare agents. In some embodiments, the chemical warfare agent is selected from the group consisting of chlorine gas and sulfur mustard. In some embodiments, the toxic-inhalation lung injury is chlorine-induced bronchiolitis obliterans syndrome (BOS) and sulfur mustard-induced bronchiolitis obliterans syndrome (BOS).

In some embodiments, the toxic-inhalation lung injury is caused by inhalation of one or more environmental and/or industrial toxic agents. In some embodiments, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes.

In some embodiments, the toxic-inhalation lung injury is pneumoconiosis or bronchiolitis obliterans. In some embodiments, the toxic-inhalation lung injury is a vaping-associated lung injury. In some embodiments, the vaping-associated lung injury is caused by inhalation of one or more agents selected from the group consisting of diacetyl, α-Tocopheryl acetate, 2,3-pentanedione, nicotine, carbonyls, benzene, toluene, metals, bacterial endotoxins, and fungal glucans.

In some embodiments, the inflammatory disorder is an inflammatory disorder of the lung. In some embodiments, the inflammatory disorder of the respiratory tract is an inflammatory disorder of the lower airways.

In some embodiments, a sustained exposure of the pharmaceutical composition in a lung epithelial lining fluid is between about 15 hours and about 100 hours. In some embodiments, the sustained exposure of the pharmaceutical composition in the lung epithelial lining fluid is at least 24 hours.

In some embodiments, the pharmaceutical composition is administered between about once per week and about three times per day. In some embodiments, the pharmaceutical composition is administered about once or twice daily.

In some embodiments, the pharmaceutical composition is administered via inhalation for between about 3 minutes and about 20 minutes.

In some embodiments, the pharmaceutical composition is administered at a dose of between about 0.5 mg/kg and about 2 mg/kg.

In some embodiments, the pharmaceutical composition binds with substantially similar affinity as an endogenous IL-1β ligand to an IL-1 type 1 receptor.

### Pharmaceutical Compositions

This disclosure also provides a pharmaceutical composition comprising proinflammatory cytokine inhibitor (e.g., anakinra) and a pharmaceutically acceptable carrier.

In some embodiments, anakinra is administered in a pharmaceutical composition comprising anakinra and a pharmaceutically acceptable carrier. In some embodiments, the pharmaceutical composition is a spray, aerosol, gel, solution, emulsion, or suspension.

The composition is preferably administered to the mammal in a pharmaceutically acceptable carrier. Typically, in some embodiments, an appropriate amount of a pharmaceutically acceptable salt is used in the formulation to render the formulation isotonic.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of anakinra being administered.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of at least one proinflammatory cytokine inhibitor (e.g., anakinra) composition, such as a buffer, a reducing agent, a bulk protein, amino acids (such as e.g., glycine or praline) or a carbohydrate. Bulk proteins useful in formulating at least one proinflammatory cytokine inhibitor composition proteins include albumin. Typical carbohydrates useful in formulating anakinra include but are not limited to sucrose, mannitol, lactose, trehalose, or glucose.

Surfactants may also be used to prevent soluble and insoluble aggregation and/or precipitation of proteins included in the composition. Suitable surfactants include but are not limited to sorbitan trioleate, soya lecithin, and oleic acid. In certain cases, solution aerosols are preferred using solvents such as ethanol. Thus, formulation including anakinra can also include a surfactant that can reduce or prevent surface-induced aggregation of anakinra caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. Especially preferred surfactants for purposes of this invention are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called “stabilizers” or “preservatives” and may include, but are not limited to, amino acids, antioxidants, pH buffers, or salt buffers.

Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing a proinflammatory cytokine inhibitor (e.g., anakinra) at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

A pharmaceutical composition of the invention is formulated to be compatible with its intended route of administration. Examples of routes of administration within the lower airways include, but are not limited to, oral or nasal inhalation (e.g., inhalation of sufficiently small particles to be deposited expressly within the lower airways). In various embodiments, the pharmaceutical compositions or single unit dosage forms are sterile and in suitable form for administration to a subject, preferably an animal subject, more preferably a mammalian subject, and most preferably a human subject.

The composition, shape, and type of dosage forms of the invention will typically vary depending on their use. Non limiting examples of dosage forms include powders; solutions; aerosols (e.g., sprays, metered or nonmetered dose atomizers, oral or nasal inhalers including metered dose inhalers (MDI)); liquid dosage forms suitable for mucosal administration to a patient, including suspensions (e.g., aqueous or non-aqueous liquid suspensions, oil-in-water emulsions, or a water-in-oil liquid emulsions), solutions, and sterile solids (e.g., crystalline or amorphous solids) that can also be reconstituted to provide liquid dosage forms suitable for lower airways administration. Formulations in the form of powders or granulates may be prepared using the ingredients mentioned above in a conventional manner using, e.g., a mixer, a fluid bed apparatus or a spray drying equipment.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., anakinra) as an active ingredient.

A liquid composition herein can be used as such with a delivery device, or they can be used for the preparation of pharmaceutically acceptable formulations comprising anakinra that are prepared for example by the method of spray drying. The methods of spray freeze-drying proteins for pharmaceutical administration disclosed in Maa et al., Curr. Pharm. Biotechnol., 2001, 1, 283-302, are incorporated herein. In another embodiment, the liquid solutions herein are freeze spray dried and the spray-dried product is collected as a dispersible anakinra-containing powder that is therapeutically effective when administered into the lower airways of an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Dosage Forms

The current invention provides for dosage forms comprising a proinflammatory inhibitor (e.g., anakinra) suitable for treating inflammatory disorders within the lower airways. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

Generally, a dosage form used in the acute treatment of a disorder may contain larger amounts of one or more of the active ingredients it comprises than a dosage form used in the chronic treatment of the same disease. In addition, the prophylactically and therapeutically effective dosage form may vary among different types of disorders. For example, a therapeutically effective dosage form may contain a compound that has an appropriate antibacterial action when intending to treat a lower airways disorder associated with a bacterial infection. These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

Suitable excipients (e.g., carriers and diluents) and other materials that can be used to provide dosage forms encompassed by this invention are well known to those skilled in the pharmaceutical sciences, and depend on the particular tissue to which a given pharmaceutical composition or dosage form will be applied. With that in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Emulsifying agents, preservatives, antioxidants, gel-forming agents, chelating agents, moisturizers, or humectants can also be added to pharmaceutical compositions and dosage forms if desired. Examples of such additional ingredients are well known in the art. See, e.g., Remington's Pharmaceutical Sciences; Remington: The Science and Practice of Pharmacy; Pharmaceutical Dosage Forms and Drug Delivery Systems, supra.

Powders and sprays can contain, in addition to a compound of this invention (e.g., anakinra), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

In a specific embodiment, the invention provides formulations for administration to the lower airways. Typically, the composition comprises an active compound(s) in combination with vehicles or the active compound is incorporated in a suitable carrier system. Pharmaceutically inert vehicles and/or excipients for the preparation of the composition include, e.g., buffering agents such as boric acid or borates, pH adjusting agents to obtain optimal stability or solubility of the active compound, lactose as a carrier, tonicity adjusting agents such as sodium chloride or borates, viscosity adjusting agents such as hydroxypropyl cellulose, methylcellulose, polyvinylpyrrolidone, polyvinyl alcohols or polyacrylamide, oily vehicle such as vehicles comprising arachis oil, castor oil and/or mineral oil. Emulsions and suspensions of the active drug substance may also be presented in the composition. In these cases, the composition may furthermore comprise stabilizing, dispersing, wetting, emulsifying and/or suspending agents.

Additional components may be used prior to, in conjunction with, or subsequent to treatment with active ingredients of the invention (e.g., anakinra). For example, penetration enhancers can be used to assist in delivering the active ingredients to the tissue. Suitable penetration enhancers include but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); and urea.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

### Administration to a Subject

The amount of the compound or composition of the invention that will be effective in conjunction with a particular method will vary, e.g., with the nature and severity of the disorder and the device by which the active ingredient(s) is administered. The frequency and dosage will also vary according to factors specific for each subject, such as age, body, weight, response, and the past medical history of the subject. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (60th ed., 2006).

In general, the recommended daily dose range of a compound of the invention for the conditions described herein lie within the range of from about 0.01 mg to about 200 mg per day, given as a single once-a-day dose preferably or as divided doses throughout a day. In one embodiment, the daily dose is administered twice daily in equally divided doses. Specifically, a daily dose range should be from about 100 micrograms to about 50 milligrams per day, more specifically, between about 500 micrograms and about 5 milligrams per day. In managing the patient, the therapy should be initiated at a lower dose, perhaps about 500 micrograms, and increased if necessary up to about 5.0 milligrams per day as either a single dose or divided doses, depending on the patient's global response. It may be necessary to use dosages of the active ingredient outside the ranges disclosed herein in some cases, as will be apparent to those of ordinary skill in the art. Furthermore, it is noted that in instances where a clinician or treating physician is involved, such a person will know how and when to interrupt, adjust, or terminate therapy in conjunction with individual subject response.

In some embodiments, the effective amount of anakinra is from about 0.1 mg to about 100 mg per day, from about 0.1 mg to about 50 mg per day, or from about 0.1 mg to about 10 mg per day. Effective dosages and schedules for administering the composition may be determined empirically, and making such determinations is within the skill in the art. Those skilled in the art will understand that the dosage of any composition that must be administered will vary depending on, for example, the mammal which will receive the composition, the route of administration, the particular composition used including the co-administration of other drugs and other drugs being administered to the mammal. A typical daily dosage of the composition used alone might range from about e.g., 0.25 mg to up to 5.0 mg per oral or nasal inhalation, or 0.125 mg to 2.5 mg per oral or nasal inhalation, however, depending on symptoms and body weight a higher or lower dosage may be appropriate.

Alternatively, the dosage administered can vary depending upon known factors, such as the pharmacodynamic characteristics of the particular agent, and its mode and route of administration; age, and health of the recipient; nature and extent of symptoms, kind of concurrent treatment, frequency of treatment, and the effect desired. As an example, treatment of mammals can be provided as a one-time or periodic dosage of anakinra from 0.01 to 200 mg, or 0.01 to 100 mg, such as 0.025, 0.05, 0.075, 0.1, 0.125, 0.25, 0.50, 0.75, 1.0, 1.125, 1.25, 1.5, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, 100, 110, 120, 130, 140, 150, 160, 170, 180, 190, 200 mg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses. It will furthermore be appreciated that a therapeutically effective amount of a particular composition can be determined by those of ordinary skill in the art with due consideration of the factors pertinent to the subject. In some specific embodiments, the effective amount of anakinra is from about 0.125 mg to 5.0 mg per day.

Different therapeutically effective amounts of a specific composition may be applicable for different diseases, as will be readily known by those of skill in the art. Similarly, different therapeutically effective compounds may be included in a specific composition depending on the subject's disease. Similarly, amounts sufficient to prevent, manage, treat or ameliorate such disorders, but insufficient to cause, or sufficient to reduce, adverse effects associated with the compounds of the invention are also encompassed by the above described dosage amounts and dose frequency schedules. Further, when a subject is administered multiple dosages of a compound or compositions of the invention, not all of the dosages need be the same. For example, the dosage administered to the subject may be increased to improve the prophylactic or therapeutic effect of the compound or it may be decreased to reduce one or more side effects that a particular subject is experiencing.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, where a physician or clinical visit is involved, two or more therapies (e.g., prophylactic or therapeutic agents) are administered within the same subject visit. The therapies can be administered simultaneously.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing or treating a disorder, (e.g., lower airways inflammatory disorders, or symptoms thereof) comprising administering to a subject in need thereof a dose of at least 100 micrograms, preferably at least 250 micrograms, at least 500 micrograms, at least 1000 micrograms, at least 5000 micrograms, or more of one or more compounds of the invention once every 3 days, preferably, once every 4 days, once every 5 days, once every 6 days, once every 7 days, once every 8 days, once every 10 days, once every two weeks, once every three weeks, or once a month.

### Articles of Manufacture

The invention encompasses articles of manufacture. A typical article of manufacture of the invention comprises a unit dosage form of a composition or compound of the invention. In one embodiment, the unit dosage form is a container, preferably a sterile container, containing an effective amount of a composition or compound of the invention and a pharmaceutically acceptable carrier or excipient. The article of manufacture can further comprise a label or printed instructions regarding the use of composition or compound or other informational material that advises the physician, technician, consumer, subject, or patient on how to prevent, treat or derive beneficial result pertaining to the disorder in question. The article of manufacture can include instructions indicating or suggesting a dosing regimen including, but not limited to, actual doses, monitoring procedures, and other monitoring information. The article of manufacture can also further comprise a unit dosage form of another prophylactic or therapeutic agent, for example, a container containing an effective amount of another prophylactic or therapeutic agent. In a specific embodiment, the article of manufacture comprises a container containing an effective amount of a composition or compound of the invention and a pharmaceutically acceptable carrier or excipient and a container containing an effective amount of another prophylactic or therapeutic agent and a pharmaceutically acceptable carrier or excipient. Examples of other prophylactic or therapeutic agents include, but are not limited to, those listed above. Preferably, the packaging material and container included in the article of manufacture are designed to protect the stability of the product during storage and shipment.

Article of manufacture of the invention can further comprise devices that are useful for administering the unit dosage forms. Examples of such devices include, but are not limited to, syringes, dry powder inhalers, metered dose and nonmetered dose inhalers, and nebulizers.

Articles of manufacture of the invention can further comprise pharmaceutically acceptable vehicles or consumable vehicles that can be used to administer one or more active ingredients (e.g., a compound of the invention). For example, if an active ingredient is provided in a solid form that is to be reconstituted for lower airways administration, the article of manufacture can comprise a sealed container of a suitable vehicle in which the active ingredient can be dissolved. For lower airways administration, a particulate-free sterile solution is preferred. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In another embodiment of the invention, articles of manufacture and kits are provided containing materials useful for treating the pathological conditions described herein and associated problems. The article of manufacture comprises a container with a label. Suitable containers include, for example, bottles, vials, and test tubes. The containers may be formed from a variety of materials such as glass or plastic. The container holds a composition having at least one active compound that is effective for treating, for example, inflammatory disorders. The active agent in the composition is a proinflammatory cytokine inhibitor, and the composition may contain one or more active agents. The label on the container indicates that the compositions is used for treating, for example, lower airways inflammatory disorders, and may indicate directions for in vivo use, such as those described above.

In some embodiments, articles of manufacture and kits are provided that specifically incorporate an inhaler. The inhaler preferably is effective at delivering a compound or composition of the invention to specific sites within the lower airways, while minimizing drug distribution to the pharynx and upper airways. The delivery device may incorporate certain parts including but not limited to filters, needles, syringes, valves, atomizers, nasal adapters, electronic nebulizers, meters, heating elements, reservoirs, a power source(s); and package inserts with instructions for use.

The kit of the invention comprises the container described above and may also include a second or third container comprising a pharmaceutically acceptable carrier or buffer, dosing reservoir, or a surfactant. It may further include other materials desirable from a commercial and user standpoint, including other buffers, diluents, and a device for delivery expressly to the lower airways incorporating filters, needles, syringes, valves, atomizers; and package inserts with instructions for use.

### Delivery Devices

A general aspect of the current invention is the local delivery expressly to the lower airways of the composition and the delivery device that accomplishes said dosing. Delivery devices of the current invention provide methods for the local delivery of the composition whereby one or more pharmacologically active agents or local treatments of the composition may have local effects expressly in the vicinity of the mucosa of the lower airways. The advantages of local therapy for local disease include the lack of adverse effects due to systemic exposure of the active ingredient.

For pulmonary administration, preferably at least one proinflammatory cytokine inhibitor (e.g., anakinra) is delivered in a particle size effective for reaching the lower airways. There are a several desirable features of an inhalation device for administering the proinflammatory cytokine inhibitors and compositions of the present invention. To be specific, delivery by the inhalation device is generally reliable, reproducible, and accurate. The inhalation device can optionally deliver small dry particles, e.g., less than about 10 microns, preferably about 3 to 5 microns, for good respirability, or dry particles with small stokes radius.

According to the invention, at least one pro-inflammatory cytokine inhibitor (e.g., anakinra) can be delivered by any of a variety of inhalation devices known in the art for administration of a therapeutic agent by inhalation. These devices capable of depositing aerosolized formulations in the lower airways of a patient include but are not limited to metered dose inhalers, sprayers, nebulizers, and dry powder generators. Other devices suitable for pulmonary administration of proteins and small molecules, including proinflammatory cytokine inhibitors, are also known in the art. All such devices can use of formulations suitable for the dispensing of proinflammatory cytokine inhibitors in an aerosol. Such aerosols can include nanoparticles, microparticles, solutions (both aqueous and nonaqueous), or solid particles.

Nebulizers like AERx Aradigm, the Ultravent nebulizer (Mallinckrodt), and the Acorn II nebulizer (Marquest Medical Products) (U.S. Pat. No. 5,404,871, WO 97/22376, entirely incorporated herein by reference), produce aerosols from solutions. In some embodiments, the nebulizer is Monaghan Aeroeclipse II Breath Activated Jet Nebulizer, or the Philips Innospire Go vibrating mesh nebulizer. In some embodiments, the nebulizer is a next-generation Philips device that use the same mesh, such as the iNeb AAD and the iNeb Advance. iNeb AAD is in labeled use in the U.S. for Ventavis (Actelion) and in the EU for Promixin (colistin for CF), both are exclusive within indication, and are in the label.

Metered dose inhalers such as the Ventolin metered dose inhaler, typically use a propellent gas and require actuation during inspiration (see, e.g., WO 94/16970, WO 98/35888, entirely incorporated herein by reference).

Suitable dry powder inhalers like Turbuhaler (Astra), Rotahaler (Glaxo), Diskus (Glaxo), Spiros inhaler (Dura/Elan) devices, the Spinhaler powder inhaler (Fisons), InnoSpire Go mesh nebulizer (Philips), iNeb AAD system (Philips) the iNeb Advance nebulizer (Philips), and PARI nebulizer (PARI), use breath-actuation of a mixed powder (U.S. Pat. No. 4,668,218, EP 237507, WO 97/25086, WO 94/08552, U.S. Pat. No. 5,458,135, WO 94/06498, all of which are herein entirely incorporated by reference). Metered dose inhalers, dry powder inhalers and the like generate small particle aerosols.

These specific examples of commercially available inhalation devices are intended to be a representative of specific devices suitable for the practice of this invention and are not intended as limiting the scope of the invention. In some embodiments, a composition comprising at least one proinflammatory cytokine inhibitor (e.g., anakinra) is delivered by a dry powder inhaler or a sprayer. In other embodiments, a composition comprising at least one proinflammatory cytokine inhibitor (e.g., anakinra) is an aerosolized formulation delivered by an aerosolized nebulizer.

Once more, the composition of the present invention can be administered as a topical spray or powder to the lower airways of a mammal by a delivery device (e.g., oral or nasal inhaler, aerosol generator, oral dry powder inhaler, through a fiberoptic scope, or via syringe during surgical intervention). These numerous drug delivery devices capable of drug distribution to the lower airways can use a liquid, semisolid, and solid composition. Investigators have found the site of deposition in the lower airways and the deposition area depend on several parameters related to the delivery device, such as mode of administration, particle size of the formulation and velocity of the delivered particles. They describe several in vitro and in vivo methods that may be used by one of ordinary skill in the art to study distribution and clearance of therapeutics delivered to the lower airways, all of which is incorporated in its entirety, herein. Thus, any of these devices may be selected for use in the current invention, given one or more advantages for a particular indication, technique, and subject. These delivery devices include but are not limited to devices producing aerosols (metered-dose inhalers (MDIs)), nebulizers and other metered and nonmetered inhalers.

In general, current container-closure system designs for inhalation spray drug products include both premetered and device-metered presentations using mechanical or power assistance and/or energy from patient inspiration for production of the spray plume. Premetered presentations may contain previously measured doses or a dose fraction in some type of units (e.g., single, multiple blisters, or other cavities) that are subsequently inserted into the device during manufacture or by the patient before use. Typical device-metered units have a reservoir containing formulation sufficient for multiple doses that are delivered as metered sprays by the device itself when activated by the patient.

An embodiment of the current invention is the use of a delivery device that is able to distribute the composition expressly to the mucosa of the lower airways in a subject in need of such treatment. In some embodiments, the delivery device is able to distribute the composition expressly to the mucosa of the lower airways in a subject in need of such treatment, with a small amount of composition reaching the pharynx and upper airways. In some embodiments, the delivery device is able to distribute the composition expressly to the mucosa of the lower airways in a subject in need of such treatment, with a minimal amount distributed to the posterior pharynx and the upper airways. In some embodiments, the delivery device is able to distribute the composition expressly to the mucosa of the lower airways in a subject in need of such treatment, with a negligible amount distributed to the posterior pharynx and the upper airways.

The current invention also incorporates multidose metering or nonmetering inhalers that are especially suited for repeated administrations and can provide numerous doses (typically 60 to up to about 130 doses, or more) either with or without stabilizers and preservatives.

Administration of a composition comprised of a proinflammatory cytokine inhibitor (e.g., anakinra) as a spray can be produced by forcing a suspension or solution of at least one proinflammatory cytokine inhibitor through a nozzle under pressure. The nozzle size and configuration, the applied pressure, and the liquid feed rate can be chosen to achieve the desired output and particle size to optimize deposition expressly in the lower airways. An electrospray can be produced, for example, by an electric field in connection with a capillary or nozzle feed. Advantageously, particles of at least one proinflammatory cytokine inhibitor composition delivered by a sprayer have a particle size less than about 20 microns, preferably in the range below 10 microns, and most preferably, about 3 to 5 microns, but other particle sizes may be appropriate depending on the device, composition, and subject needs.

Commercially available nebulizers for liquid formulations, including jet nebulizers and ultrasonic nebulizers may also be useful for administration to the lower airways. Liquid formulations may be directly nebulized and lyophilized powder nebulized after reconstitution. Alternatively, the composition may be aerosolized using a metered dose inhaler, or inhaled as a lyophilized and milled powder. In addition, the liquid formulation of composition may be instilled through a bronchoscope, placed directly into the affected regions.

In one embodiment of the present invention, a proinflammatory cytokine inhibitor (e.g., anakinra) may be administered by a metered dose inhaler. The metered-dose inhaler can contain therapeutically active ingredients dissolved or suspended in a propellant, a mixture of propellants, or a mixture of solvents, propellants, and/or other excipients in compact pressurized aerosol dispensers. The MDI may discharge up to several hundred metered doses of the composition. Depending on the composition, each actuation may contain from a few micrograms (μg) up to milligrams (mg) of the active ingredients delivered in a volume typically between 25 and 100 micro liters. In a metered dose inhaler (MDI), a propellant, at least one proinflammatory cytokine inhibitor (e.g., anakinra), and various excipients or other compounds are contained in a canister as a mixture including a liquified compressed gas (propellants).

Actuation of the metering valve releases the mixture as an aerosol, preferably containing particles in the size range of less than about 20 microns. In some embodiments, the particle size is less than about 10 microns. In some embodiments, the particle size is below 5 microns. The desired aerosol particle size can be obtained by employing a formulation of antibody composition protein produced by various methods known to those of skill in the art, including jet-milling, spray drying, critical point condensation, or other methods well known to one of ordinary skill in the art.

Compositions of at least one proinflammatory cytokine inhibitor (e.g., anakinra) for use with a metered-dose inhaler device can include a finely divided powder containing at least one proinflammatory cytokine inhibitor as a suspension in a non-aqueous medium, for example, suspended in a propellant with the aid of a surfactant or solubilizing agent. The propellant can be any conventional material including but not limited to chlorofluorocarbon, a hydrochlorofluorocarbon, a hydrofluorocarbon, or a hydrocarbon, including trichlorofluoromethane, dichlorodifluoromethane, dichlorotetrafluoroethanol and 1, 1,1,2-tetrafluoroethane, HFA-134a (hydrofluroalkane-134a), HFA-227 (hydrofluroalkane-227). Hydrofluorocarbon is a preferred propellant. A surfactant can be chosen to stabilize the at least one proinflammatory cytokine inhibitor as a suspension in the propellant, to protect the active agent against chemical degradation. In some cases, solution aerosols are preferred using solvents such as ethanol for more water-soluble active agents. Additional agents including a protein can also be included in the composition.

One of ordinary skill in the art will recognize that the methods of the current invention can be achieved by lower airways administration of at least one proinflammatory cytokine inhibitor (e.g., anakinra) compositions via devices not described herein. The current invention also incorporates unit-dose metering and nonmetering spray devices that are especially suited for single administration. These devices are typically used for acute short-term treatments (i.e., acute exacerbations) and single-dose delivery (i.e., long acting compositions) and can accommodate a liquid, powder, or mixture of both formulations of the composition. However, in certain circumstances, these unit dose devices may be preferred over multidose devices when used repeatedly in a particular way. Such uses may include but are not limited to repeated procedures where a sterile device is preferred.

Another embodiment of the invention provides for a single-dose syringe prefilled with the composition appropriate for treating the lower airways inflammatory disease of the subject. Said prefilled syringe may be sterile or nonsterile and used in dose administration during procedures to a subject in need of lower airways therapy. An example of an application where a syringe is preferred includes, but is not limited to, the distribution of composition through an endoscope. These examples are not intended to be limiting and one skilled in the art will appreciate that other options exist for delivery of the composition expressly to the lower airways and these are incorporated herein.

In one embodiment of the invention, a composition containing one or more therapeutic agents described herein is directly administered to the lower airways. Such administration may be carried out via use of an intrapulmonary aerolizer, which create an aerosol containing the composition and which may be directly installed into the lower airways. Exemplary aerolizers are disclosed in U.S. Pat. Nos. 5,579,578; 6,041,775; 6,029,657; 6,016,800, and 5,594,987, all of which are herein incorporated by reference in their entirety. Such aerolizers are small enough in size so they can be inserted directly into the lower airways, for example into an endotracheal tube or even into the trachea. In one embodiment, the aerolizer may be positioned near the carina, or first bifurcation, of the lung. In another embodiment, the aerolizer is positioned so as to target a specific area of the lung, for example an individual bronchus, bronchiole, or lobe. Since the spray of the device is directly introduced into the lungs, losses due to deposition of the aerosol due to deposition on the walls of the nasal passages, mouth, throat, and trachea are avoided. Optimally, the droplet size produced by such suitable aerolizer is somewhat larger than those produced by ultrasonic nebulizers. Therefore, the droplets are less likely to be exhaled and thus leading to a delivery efficiency of virtually 100%. In addition, the delivery of the compositions has a highly uniform pattern of distribution.

In one embodiment, such an intrapulmonary aerosolizer comprises an aerosolizer attached to a pressure generator for delivery of liquid as an aerosol and which can be positioned in close proximity to the lungs by being inserted into the trachea directly or into an endotracheal tube or bronchoscope positioned within the trachea. Such an aerolizer may operate at pressures of up to about 2000 psi and produces particles with a medium particle size of 12 μm.

In an alternate embodiment, such an intrapulmonary aerosolizer comprises a substantially elongated sleeve member, a substantially elongated insert, and a substantially elongated body member. The sleeve member includes a threaded inner surface, which is adapted to receive the insert, which is a correspondingly threaded member. The threaded insert provides a substantially helical channel. The body member includes a cavity on its first end, which terminates by an end wall at its second end. The end wall includes an orifice extending therethrough. The body member is connected with the sleeve member to provide the aerosolizer of the present invention. The aerosolizer is sized to accommodate insertion into the trachea of a subject for administration of compositions containing anakinra. For operation of the device, the aerosolizer is connected by a suitable tube with a liquid pressure driver apparatus. The liquid pressure driver apparatus is adapted to pass liquid material (e.g., a composition containing one or more proinflammatory cytokine inhibitor) therefrom which is sprayed from the aerosolizer. Due to the location of the device deep within the trachea, the liquid material is sprayed in close proximity to the lungs, with resulting improved penetration and distribution of the sprayed material in the lungs.

In an alternate embodiment, such an aerosolizer, sized for intratracheal insertion, is adapted for spraying a composition containing anakinra directly into the lower airways (e.g., in close proximity to the lungs). The aerosolizer is placed into connection with a liquid pressure driver apparatus for delivering of the liquid composition. The aerosolizer comprises a generally elongated sleeve member, which defines a first end and a second end and includes a longitudinally extending opening therethrough. The first end of the sleeve member is placed in connection with the liquid pressure driver apparatus. A generally elongated insert is also provided. The generally elongated insert defines a first end and a second end and is received within at least a portion of the longitudinally extending opening of the sleeve member. The insert includes an outer surface which has at least one substantially helical channel provided surrounding its outer surface which extends from the first end to the second end. The substantially helical channel of the insert is adapted to pass the liquid material, which is received by the sleeve member. A generally elongated body member is also included which is in connection with the sleeve member. The body member includes a cavity provided in its first end, which terminates at an end wall which is adjacent its second end. The end wall is provided having an orifice therein for spraying the liquid material, which is received from the insert. The portions of the sleeve member, insert and body member, in combination, are of sufficient size to allow for intratracheal insertion. A method of using such an aerosolizer includes the steps of connecting an aerosolizer with a first end of a hollow tube member and connecting the second end of the hollow tube member with the liquid pressure driver apparatus. The method further includes the steps of providing the aerosolizer in the trachea or into a member which is provided in the trachea, and then activating the liquid pressure driver apparatus for spraying a composition containing one or more proinflammatory cytokine inhibitors therefrom.

In an alternate embodiment, a powder dose composition containing one or more proinflammatory cytokine inhibitors is directly administered to the lower airways via use of a powder dispenser. Exemplary powder dispensers are disclosed in U.S. Pat. Nos. 5,513,630, 5,570,686 and 5,542,412, all of which are herein incorporated in their entirety. Such a powder dispenser is adapted to be brought into connection with an actuator, which introduces an amount of a gas for dispensing the powder dose. The dispenser includes a chamber for receiving the powder dose and a valve for permitting passage of the powder dose only when the actuator introduces the gas into the dispenser. The powder dose is passed from the dispenser via a tube to the lower airways of the subject. The powder dose may be delivered intratracheally, near the carina, which bypasses the potential for large losses of the powder dose to e.g., the mouth, throat, and trachea. In addition, in operation the gas passed from the actuator serves to slightly insufflate the lungs, which provides increased powder penetration. For the intratracheal insertion, the tube can be effected through an endotracheal tube in anesthetized, ventilated subjects, including animal or human patients, or in conscious subjects, the tube be inserted directly into the trachea preferably using a small dose of local anesthetic to the throat and/or a small amount of anesthetic on the tip of the tube, in order to minimize a “gag” response.

In one embodiment, a composition containing one or more therapeutic agents described herein is directly administered to the lower airways. Such administration may be carried out via use of an aerolizer, which create an aerosol containing the composition and which may be directly installed into the lower airways. Exemplary aerolizers are disclosed in U.S. Pat. Nos. 5,579,758; 6,041,775; 6,029,657; 6,016,800; 5,606,789; and 5,594,987 all of which are herein incorporated by reference in their entirety. The invention thus provides for the methods of administering compositions containing one or more proinflammatory cytokine inhibitors directly to the lower airways by an aerolizer.

In particular, an embodiment of the present invention is a new use for the “intratracheal aerosolizer” device which methodology involves the generation of a fine aerosol at the tip of a long, relatively thin tube that is suitable for insertion into the trachea. Thus, the present invention provides a new method of use for this aerosolizer technology in a microcatheter as adapted herein, for use in the lower airways in the prevention, treatment, and care of lower airways disorders.

In another embodiment of the invention, an aerosolizing microcatheter is used to administer a composition containing pro-inflammatory cytokine inhibitor. Examples of such catheters and their use, termed “intratracheal aerosolization,” which involves the generation of a fine aerosol at the tip of a long, relatively thin tube that is suitable for insertion into the trachea, are disclosed in U.S. Pat. Nos. 5,579,758; 5,594,987; 5,606,789; 6,016,800; and 6,041,775.

In a further embodiment, a new use for the microcatheter aerosolizer device (U.S. Pat. Nos. 6,016,800 and 6,029,657) is adapted for nasal and paranasal sinus delivery and uses to deliver bioactive agents (e.g., anakinra) in the treatment, prevention, and diagnosis of lower airways disorders. One advantage of this microcatheter aerosolizer is the potential small size (0.014″ in diameter), and thus capable of being easily inserted into the working channel of a human flexible (1 to 2 mm in diameter) or ridged endoscope and thereby directed partially or completely into the ostium of a paranasal sinus.

One of ordinary skill in the art will recognize that the methods of the current invention can be achieved by lower airways administration of anakinra composition via devices not described herein.

### Uses of the Compositions and Compounds of the Invention

The present invention provides methods for preventing, managing, treating, or ameliorating a disorder (e.g., inflammatory disorders of the lower airways) using a compound (e.g., anakinra) or composition of the invention in combination with another modality, such as a prophylactic or therapeutic agent known to be useful for, or having been or currently being used in the prevention, treatment, management, or amelioration of a disorder or used in the lessening of discomfort or pain associated with a disorder. Depending on the manner of use of the compositions or compounds, the invention can be co-administered with another modality, or the compositions or compounds of the invention can be mixed and then administered as a single composition to a subject. It is of course contemplated that the methods of the invention can be employed in combination with still other therapeutic uses such as surgical resection and lung transplantation.

In the methods, a composition is administered to a mammal diagnosed as having inflammatory disorder(s) of the lower airways. It is of course contemplated that the methods of the invention can be employed in combination with still other therapeutic techniques such as endoscopic monitoring and treatment techniques, surgical resection and lung transplantation.

Described herein are uses of the compounds and compositions of the invention for attaining a beneficial effect pertaining to lower airways inflammatory disorders, providing a beneficial effect pertaining to such disorders, or one or more symptoms thereof. The methods comprise administering to a subject in need thereof a prophylactically or therapeutically effective amount of, one or more compounds or a composition(s) of the invention. For example, administration of such compounds can be via one or more of the pharmaceutical compositions of the invention. It is of course contemplated that the methods of the invention can be employed in combination with oral or nasal inhalation devices. Importantly, it is contemplated that the methods of the invention can be employed in combination with the subcutaneous or intravenous injection, or other systemic routes of administration of proinflammatory cytokine inhibitors (e.g., anakinra). However, still other therapeutic techniques such as endoscopic procedures and treatment techniques, surgical resection and lung transplantation are all included herein.

In one embodiment, a subject in need of prevention, treatment, management, or amelioration of a disorder or a symptom thereof is a subject that has the disorder, that is known to be at risk of the disorder, has been diagnosed with the disorder, has previously recovered from the disorder, or is resistant to current therapy. In particular embodiments, the subject is an animal, preferably a mammal, and more preferably a human, that is predisposed and/or at risk because of a genetic factor(s), an environmental factor(s), or a combination thereof to develop the disorder. In yet another embodiment, the subject is refractory or non-responsive to one or more other treatments for a disorder. In yet another embodiment, the subject is an immunocompromised or immunosuppressed mammal, such as a human.

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—ALTA-2530 Target Product Attributes (GLP/GMP)

In some embodiments, ALTA-2530 formulations have the product attributes as described herein. In some embodiments, ALTA-2530 is a solution for nebulization delivered using a Philips InnoSpire Go vibrating mesh (VM) nebulizer. In some embodiments, ALTA-2530 is a solution for nebulization that will be delivered using a preclinical nebulizer (Aerogen Solo VM nebulizer). In some embodiments, ALTA-2530 is an extemporaneously prepared solution formulation for nebulization that can be produced at the preclinical and clinical study sites and is stable to nebulization over the dosing period and a minimum in-use period of 24 hours. In some embodiments, ALTA-2530 has nebulization storage condition requiring refrigeration. In some embodiments, ALTA-2530 developed for GLP toxicology and GMP clinical studies will preferably be the same or comparable to avoid any bridging studies (e.g, excipients will not differ, and ratios will not exceed GLP qualification levels). In some embodiments, the impurity profiles of the nebulized GMP clinical formulation will be similar to and will not exceed the impurity limits qualified in the GLP preclinical studies. In some embodiments, the clinical formulation solution concentration(s) are suitable for delivering 10 -40 mg from the VM nebulizer (expressed as drug charge to nebulizer) in less than 5 minutes and ideally within 2-3 minutes using the Philips InnoSpire Go nebulizer. In some embodiments, ALTA-2530 has reproducible delivered and pulmonary lung dose to support the clinical programs as demonstrated by chemical and aerosol performance stability over the in-use period and anticipated dosing duration.

Table 1 shows a summary of key CMC activities and deliverables.

TABLE 1
CMC Activities and Deliverables

| CMC Activity | Key Deliverables |
|---|---|
| Analytical Method Development | Develop/optimize and qualify/validate phase |
| | appropriate analytical methods to support |
| | GLP; preclinical and GMP Phase 1 clinical |
| | programs for an ALTA-2530 solution for |
| | nebulization; |
| | Generate the associated analytical protocols |
| | and method development/validation reports; |
| | Generate final analytical methods; |
| | Provide supporting data for the |
| | extemporaneous product specification |
| Inhalation Formulation Screening | Identify potential protein formulation matrices |
| | to be used in a preclinical tolerability study |
| | with the Kineret matrix as a control using |
| | freeze/thaw studies; |
| | Identify preclinical formulations based on |
| | short-term stressed stability studies and the |
| | preclinical tolerability results; |
| | Conduct filter compatibility studies on lead |
| | and back-up formulations |
| Aerosol Characterization | Identify GLP (and GMP) solutions for |
| | nebulization that are stable to nebulization |
| | over the in-use and delivery periods using the |
| | preclinical nebulizer (Aerogen Solo) and the |
| | clinical nebulizer (InnoSpire Go); |
| | Generate preparation instructions to be used by |
| | the preclinical site compounding pharmacy |
| | (e.g., a pharmacy manual); |
| | Specify the in-use period and storage |
| | conditions for the prepared solutions for |
| | nebulization; |
| | Compare impurity profiles from both |
| | nebulizers and provide findings in a QA |
| | reviewed development report |
| Pulmonary Dose Confirmation and In-Use | Estimate the pulmonary dose obtained from |
| Stability Period -Extemporaneous Preparations | nebulized solutions of Kineret dilutions using |
| | the AeroEclipse II breath-activated jet |
| | nebulizer and InnoSpire Go nebulizer; |
| | Estimate the pulmonary dose and dose |
| | variability from nebulized solutions of ALTA- |
| | 2530 using the Philips InnoSpire Go nebulizer |
| | over the in-use period and dosing duration |

### Example 2—ALTA-2530 Formulations

Table 2 shows different possible embodiments for ALTA-2530 formulations.

TABLE 2
Formulation Compositions

| | | | | Role in |
|---|---|---|---|---|
| Formulation | | | | anakinra |
| Components | Component | Options | Concentration Ranges | formulations |
| Stabilizers | Surfactants | Polysorbate 80 | 0.1% | Non-ionic |
| | | Polysorbate 20 | (0.01-1%) | surfactant which |
| | | Brig 35 | | reduces |
| | | | | aggregation and |
| | | | | denaturation, |
| | | | | increases |
| | | | | solubility |
| | Chelating | EDTA di sodium | 0.5 mM (0.05-1 mM) | Stabilization |
| | agents | | | |
| | Sugars | Trehalose | >40% w/v | Conformational |
| | | Sucrose | | stability |
| | | Glycerol | | |
| | | Sorbitol | | |
| Buffers - | Buffers | Citrate | 20 mM | Combined with |
| pH~6.5-7.5 | | Phosphate | 10 mM (1-50 mM) | mannitol |
| | | Histidine | 10 mM (5-50 mM) | Combined with |
| | | Glutamate | 10 mM (10-50 mM) | taurine and HP |
| | | Pyrophosphate | | |
| | | HEPES | | |
| Tonicity | Tonicity | Sodium chloride | 140 mM (120-180 mM) | Combined with |
| modifiers | agents | Mannitol | 10 mg/mL (5-50 mg/mL) | phosphate; No |
| | Osmolytes | Taurine | 30 mg/mL (15-25 mg/mL) | Irritation |
| | | Hydroxyproline | 26 mg/mL (15-26 mg/mL) | Combined with |
| | | Proline | | HEPES |
| | | | | Combined with |
| | | | | HEPES |

Table 3 shows different possible excipients for various embodiments for ALTA-2530 formulations.

TABLE 3
Excipient Options

| | | Stabilizers | | | | Tonicity |
|---|---|---|---|---|---|---|
| | Buffers | Sugars | Chelators | Surfactants | Osmolytes | Agents |
| Formulation | Citrate | Trehalose | EDTA | Polysorbate | Taurine | NaCl |
| Components | Phosphate | Sucrose | disodium | 80 | Hydroxyproline | |
| | HEPES | Mannitol | | Polysorbate | | |
| | Glutamate | | | 20 | | |
| | | | | Sorbitan | | |
| | | | | trioleate | | |

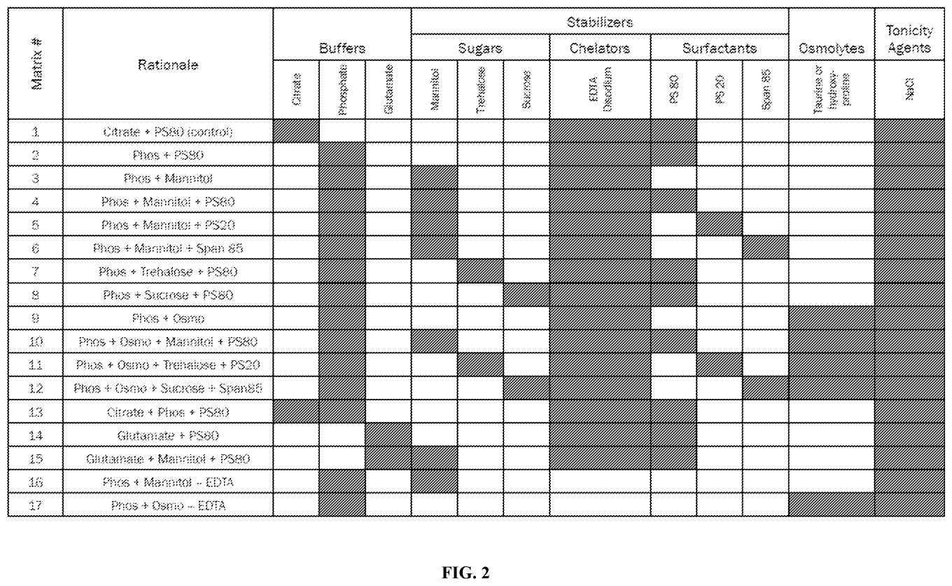

FIG. 2 shows a checkerboard for various embodiments of ALTA-2530 formulations.

### Example 3—Analytical Development

In some embodiments, key deliverables include develop/optimize and qualify/validate phase appropriate analytical methods to support GLP preclinical and GMP Phase 1 clinical programs for an ALTA-2530 solution for nebulization. All qualification/validation studies will be conducted in accordance with ICH guidelines.

In some embodiments, attributes and methods for product specification/stability include: Appearance, pH, osmolality; identification by peptide mapping; protein concentration by A280; purity by RPHPLC, SE-HPLC, reduced and non-reduced CE-SDS, and IEX-HPLC; foreign and particulate matter and subvisible particles; aerosol particle size distribution by NGI for a nebulized solution as listed in USP 601 using an appropriate duration of testing; delivered dose using breath simulator as listed in USP 1601 and USP 601 over the entire duration of dosing; Bioburden and endotoxin; Cell-based Bioassay for potency.

In some embodiments, information only test methods to support formulation development include: Circular dichroism, viscosity, surface tension, formulation density, droplet size and distribution by Malvern Spraytec or equivalent, dynamic light scattering (DLS), and turbidity. In some embodiments, support specifications for active and placebo extemporaneous products are obtained. In some embodiments, validation summary reports for all validated method activities are produced.

### Example 4—Inhalation Formulation Screening

To screen the inhalation formulations, the acceptable targeted solution formulation pH for the pulmonary route of administration is between pH 5-8. The solution osmolality is within physiological ranges (˜300 mOsm/kg). Excipients used are “acceptable” or “well characterized” by the pulmonary route and within the concentration ranges/doses listed within the FDA Inactive Ingredient List for approved pulmonary products. Preference is given to either parenteral grade excipients (if available) and/or inhalation grade excipients currently used in marketed products for inhalation in major markets, including the US, EU, and Japan. A tiered approach is used to evaluate the preformulations including a physical stability screening study, stress stability screening study, and a formulation filtration study. For preformulation screening, studies will be conducted to identify formulation matrices and stable ALTA-2530 solutions for nebulization to be used in aerosol characterization studies. A control formulation (Kineret) will be used as a reference throughout these studies.

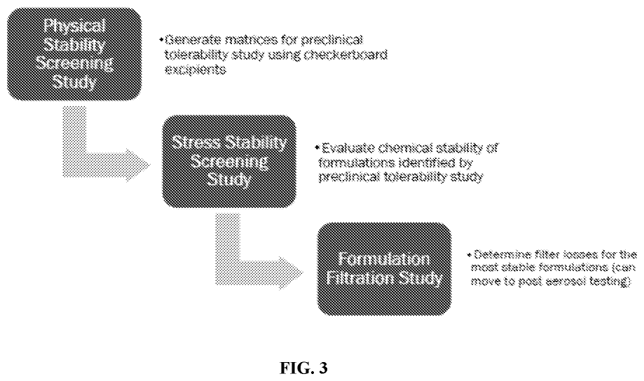

Preformulation studies are conducted in accordance with a tiered approach as shown in FIG. 3. In conducting the preformulation studies, screening studies are conducted to identify formulation matrices and stable ALTA-2530 solutions for nebulization to be used in aerosol characterization studies. ALTA-2530 is a human recombinant IL-1 receptor inhibitor (rhIL-1Ra). In some embodiments, ALTA-2530 (previously OSP-101) is a novel, inhaled formulation of rhIL-1Ra to be delivered via an approved Philips nebulizer. A control formulation (Kineret) is used as a reference. Kineret is approved for rheumatoid arthritis (USA, EU, Canada, Australia, etc.) and cryopyrin-associated periodic syndromes (up to 100 mg/day, subcutaneous injection). Formulation components include, but are not be limited to buffers, stabilizers, and tonicity modifiers (see FIG. 2). The buffers include histidine, phosphate, succinate, glutamate, citrate, PBS, and pyrophosphate. The stabilizers include polysorbate 20 and 80 and other compatible nonionic surfactants, EDTA disodium, glycerin, mannitol, and trehalose. The tonicity modifiers include sodium chloride and dextrose.

In conducting physical stability screening studies, approximately 10 formulations (various matrices+ALTA-2530 plus a Kineret control) using stressed conditions (e.g., freeze/thaw, agitation) are screened to identify potential protein formulation matrices to be used in a preclinical tolerability study. Characterization and output include physical and chemical characterization analyses of approximately 10 formulations (with ALTA-2530) (i.e., appearance, related substances, SEC, DSC, turbidity, DLS) after 1 to 2 freeze/thaw exposure(s) and agitation cycles.

In conducting stress stability screening studies, a solution formulation for use in GLP studies is evaluated using short term temperature/time stress-based stability.

In conducting formulation filtration studies, a lead and a back-up formulation is identified (up to 4 compositions; 2 matrices×2 concentrations) in the stressed testing screening studies. Filter compatibility studies (i.e., impurities and loss of content) using a maximum of 2×0.2 μm filter types is conducted. Results are generated using single and double filtration.

For physical stability screening studies, key deliverables include screening approximately 10 formulations (various matrices+ALTA-2530, Kineret control) using stressed conditions (e.g., freeze/thaw, agitation) to identify potential protein formulation matrices to be used in a preclinical tolerability study with the Kineret matrix as a control.

In some embodiments, characterization and output includes physical and chemical characterization analyses of approximately 10 formulations (with ALTA-2530) (i.e., appearance, related substances, SEC, DSC, turbidity, DLS) after 1 to 2 freeze/thaw exposure(s) and agitation cycles. In some embodiments, data is used to identify 4-6 matrices (without ALTA-2530) that are used in a preclinical tolerability study and/or used in short term stability studies. Preparation instructions and formulation components for the identified matrix compositions (without ALTA-2530) are provided to the preclinical study site.

For stress stability screening studies, key deliverables are combined with outcomes from the preclinical tolerability study, as well as identifying a solution formulation for use in GLP studies by evaluating short term temperature/time stress-based stability. A dilution of this formulation is expected to be used in Phase 1 clinical studies. In some embodiments, 4-6 formulations (e.g., 2 concentrations of 2-3 formulations) by 2-3 storage conditions and freeze/thaw (if not conducted previously) are included; as well as initial time and 3 pull points (e.g., T=0, 24 hrs, 48 hrs, 7 days). In some embodiments, stress testing conditions are determined based on existing data (literature and physical stability screening study results).

For formulation filtration studies, key deliverables include using the lead and back-up formulations (up to 4 compositions; 2 matrices×2 concentrations) identified in the stressed testing screening studies, conduct filter compatibility studies (i.e., impurities and loss of content) using a maximum of 2×0.2 μm filter types. Results will be generated using single and double filtration.

### Example 5—Aerosol Characterization

Using formulations identified in preformulation screening studies from Example 4, stability to nebulization over the in-use period (T=0 and T=24 hr) and dosing duration to simulate clinical dosing using the InnoSpire Go nebulizer is determined. A single nebulizer charge volume for these studies is also determined. Samples are evaluated from preclinical study site engineering runs to assess stability to nebulization over the anticipated dosing duration (i.e., duration of dosing for preclinical studies (e.g., 0, 1, 3 hrs)) using the preclinical nebulizer (Aerogen Solo).

To conduct the stability to nebulization study, a minimum of 2 and maximum of 4 solutions for nebulization (as identified during formulation screening studies) using both the clinical and preclinical nebulizers (if different) are characterized. An impurity profile as generated from the preclinical nebulizer over the intended dosing duration and in-use period (samples will be provided from engineering runs conducted at the preclinical study site) is determined. The impurity profile generated from the clinical nebulizer (Philips InnoSpire Go) over the intended dosing duration and in use period is also determined. Pre-nebulization assessment of solution viscosity, density, turbidity, and surface tension data is collected and analyzed. Data is also collected for both nebulizers for each formulation at T=0 and T=24 hrs (solutions stored at refrigerated conditions) to determine: assay and impurities (pre- and post-nebulization by SEC and RP-HPLC); physical characterization (appearance and turbidity pre-and post-nebulization as collected nebulized solutions and solution remaining in nebulizer); VMD and GSD by SpraytecTM; liquid output rate (LOR); and report time to empty, sputter, or clog nebulizer as well as the approximate residual volume in the nebulizer at this timepoint.

The pulmonary dose and dose variability using a minimum of 3 Philips InnoSpire Go nebulizer units over the in-use period for a maximum of 2 formulations (low and high solution concentrations, same matrix) and at 2 nebulizer charge volumes is estimated by generating APSD and GSD from 3 nebulizers; generating DD data (n=10) at a fixed duration (pre-determined time to sputter) using USP 1601; estimating the lung dose (using DD and cut-off of 5 μm and 3.5 μm APSD) and the dose variability; and estimating the lung dose as a function of multiple nebulizer charges for each fixed nebulizer charge volume.

### Example 6—Proposed Clinical Studies of ALTA-2530 in BOS

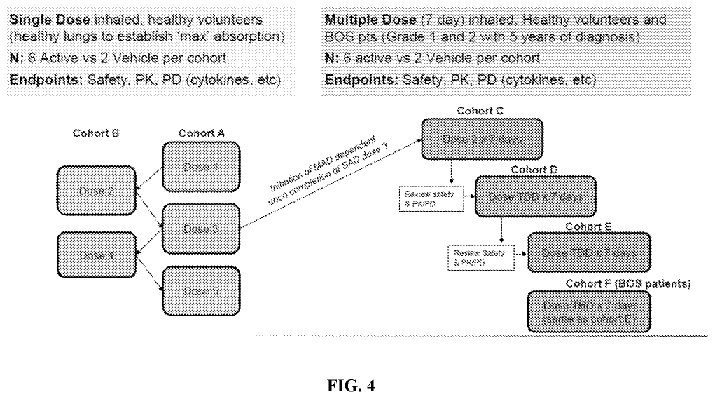

FIG. 4 shows a phase 1 study using single/multiple ascending doses (SAD/MAD) of ALTA-2530 in healthy volunteers and BOS patients. The proposed study would be conducted as single trial, with a MAD limit of 7 days in healthy volunteers and BOS patients.

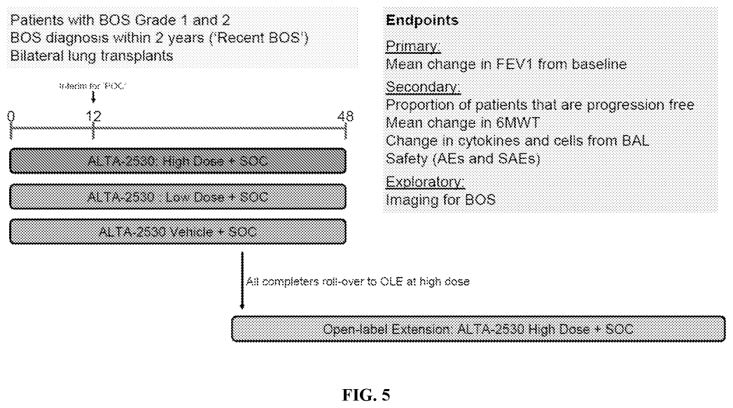

FIG. 5 shows a phase 2b/3 pivotal study of ALTA-2530 in BOS patients with 12 week POC interim.

### Example 7—Inhaled Delivery of ALTA-2530 Achieves Extensive and Prolonged Pulmonary Exposure of rhIL-1ra Compared to Low Level and Transient Exposure Following Bolus IV Injection

ALTA-2530 is a novel inhaled formulation of recombinant human IL-1 receptor antagonist (rhIL-1Ra) in development for bronchiolitis obliterans syndrome (BOS). IL-1 overexpression in BOS drives chronic inflammation and fibroblast activation leading to airway remodeling and impaired oxygen transfer. Endogenous IL-1Ra is upregulated in response to IL-1 to limit cytokine signaling, but expression is inadequate to prevent BOS. Pharmacological IL-1 blockade is considered akin to restoration of physiologic immune regulation.

### Purpose:

To determine if ALTA-2530 is stable during nebulization, achieves aerosol particle diameters consistent with distribution to distal airways, and pulmonary exposure commensurate with treatment of BOS.

### Methods:

Aerosolization and in vivo studies were performed with Aerogen Solo or Phillips InnospireGo vibrating mesh nebulizers. Rats (n=4/grp/timepoint) received ALTA-2530 by nose-only inhalation (0.63, 1.3, and 2.1 mg/g lung). Serum and bronchioalveolar lavage (BAL) samples were collected for analysis by LC-MSMS. ALTA-2530 in lung epithelial lining fluid (ELF) was calculated using a BALF dilution factor.

Inhaled delivery of ALTA-2530 achieves extensive, stable, and sustained exposure in lung epithelial lining fluid that in rodents markedly exceeds 24 hr, in contrast to exposure following bolus IV delivery where exposure is transient and <20 min. Lung is the target organ for treatment of conditions including, but not limited to: post lung transplant conditions including BOS, primary graft dysfunction (PGD), reperfusion injury, infection related ARDS, or chemical lung injury. Achieving pharmacologically relevant levels of rhIL-1Ra in lung tissue requires high-dose SC or IV treatment with rhIL-1Ra resulting in renal impairment and neutropenia in some patients. IV delivery provide low level and transient exposure to lung tissue. Inhaled delivery targets the organ of clinical significance and achieves long lasting high exposure levels.

Inhaled delivery of ALTA-2530 achieves prolonged pulmonary exposure of rhIL-1Ra that exceeded 24 hr in rat compared to transient exposure of <20 min following bolus IV injection. This is predictive for once or twice daily, or even less frequent, dosing clinically compared to multiple daily IV doses required for the treatment of lung pathologies. Moreover, the ratio of lung epithelial lining fluid to plasma exposures in rats were >2500-fold compared to 0.44-fold for lung tissue: plasma following a 5 hr IV infusion (Kim et al, Kidney as a major clearance organ for recombinant human interleukin-1 receptor antagonist, Journal of Pharmaceutical Sciences, 1995).

Recombinant human IL-1 receptor antagonist (rhIL-1Ra) exposure was determined in lung bronchioalveolar lavage fluid (BALF) following inhaled delivery to male and female Sprague Dawley rats.

Sprague Dawley male (M) and female (F) rats were weighed and randomized into study groups (Table 4). One group was kept naïve, all other animals were exposed to a single dose of either the Vehicle (normal saline, 0.9% sodium chloride), or to ALTA-2530 test article (TA) recombinant human IL-1 receptor antagonist (rhIL-1Ra) via nose-only inhalation. Target dose levels of rhIL-1Ra were regulated by exposure duration at a target aerosol concentration of 1.5 milligrams (mg)/liter (L).

TABLE 4
Experimental Design

| | | | | Target Necropsy Post Exposure | |
|---|---|---|---|---|---|
| | | Exposure | | 5, 30 minutes, 1, 2, | |
| | Target | Time | | 3, 4, 6, 8, 12 hours | 24, 72 hours |
| Group | Dose | (min) | n | (2M/2F per group) | (3M/3F per group) |
| 1 | Treatment | N/A | 3M/3F | N/A | 24 hour: TK serum, |
| | Naïve | | | | BALF, lung tissue |
| 2 | Vehicle | 180 | 3M/3F | TK serum 1 hr post- | 24 hour: TK serum, |
| | | | | exposure (non- | BALF, lung tissue |
| | | | | terminal, 3M/3F) | |
| 3 | Dose 1 | 60 | 24M/24F | TK serum, BALF, lung | TK serum, BALF, |
| | | | | tissue | lung tissue, Clinical |
| | | | | | pathology |
| 4 | Dose 2 | 120 | 24M/24F | TK serum, BALF, lung | TK serum, BALF, |
| | | | | tissue | lung tissue, Clinical |
| | | | | | pathology |
| 5 | Dose 3 | 180 | 24M/24F | TK serum, BALF, lung | TK serum, BALF, |
| | | | | tissue | lung tissue, Clinical |
| | | | | | pathology |

N/A = not applicable

Blood (serum) and BALF were collected for toxicokinetic (TK) analysis from all TK animals during scheduled necropsy following exposures.

Serum and BALF levels of rhIL-1Ra were determined by means of LC-MSMS. rhIL-1RA was captured from serum and BALF samples using streptavidin magnetic beads coated with anti-human IL-1RA antibody, subjected to “on-bead” proteolysis with trypsin, denatured, reduced, and alkylated, resulting in characteristic peptide fragments originating from rhIL-1RA. A selected characteristic peptide was quantified as a surrogate of the ALTA-2530 concentrations in samples.

Concentrations of rhIL-1Ra in BALF were corrected for the dilution factor introduced during collection of epithelial lining fluid (ELF) using normalization of BALF and plasma urea as described by Rennard et al, J Applied Physiol, 1986. Levels of urea in BALF were less than the lower limit of quantitation (LLOQ) so the normalization factor was calculated using the LLOQ value (1 mg/dL). Thus, the reported values for rhIL-1Ra in ELF are likely an under-estimate of true concentration. Mean plasma urea concentrations were used based on combined gender groups mean values for plasma urea.

### Results:

Nebulized ALTA-2530 delivered rhIL-1Ra particles with mass median aerodynamic diameters of ˜2.5-4 μm, consistent with delivery to small bronchioles. Impurity profiling by HPLC-UV and HPLC-SEC methods and an in vitro potency assay demonstrated rhIL-1Ra protein was stable during nebulization and retained full potency.

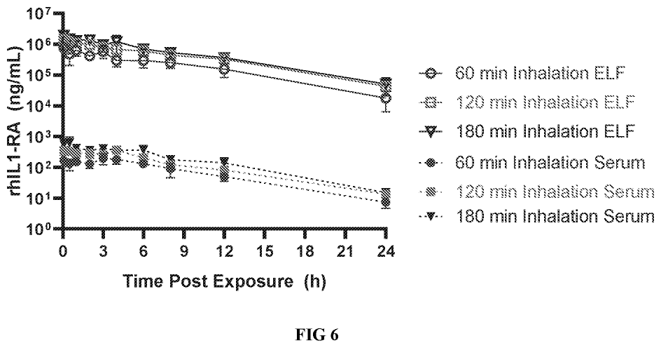

Descriptive pharmacokinetic parameters for rhIL-1Ra in serum and ELF are presented in Table 5. FIG. 6 shows concentration versus time profiles for rhIL-1Ra in ELF and serum following single doses to rat.

TABLE 5
Descriptive pharmacokinetic parameters in serum and ELF following single doses of
ALTA-2530 to rat

| Group #, | Dose ^{a} | | Tₘₐₓ | Cₘₐₓ | AUCₗₐₛₜ | AUC_{0-inf} | t_{1/2} |
|---|---|---|---|---|---|---|---|
| Dose level | (mg/kg) | Sample | (h) | (ng/mL) | (h*ng/mL) | (h*ng/mL) | (h) |
| 3, Dose 1 | 3.34 | Serum | 4.0 | 202 | 1785 | — | 4.38 |
| | | ELF | 0.08 | 754,000 | 4,990,000 | 5,430,000 | 6.36 |
| 4, Dose 2 | 7.07 | Serum | 0.08 | 400 | 3,040 | — | 4.77 |
| | | ELF | 0.08 | 1,400,000 | 9,790,000 | 1,400,000 | 5.48 |
| 5, Dose 3 | 11.20 | Serum | 0.50 | 626 | 4,525 | — | 4.14 |
| | | ELF | 0.08 | 1,920,000 | 12,500,000 | 13,700,000 | 4.21 |

^{a} Dose is average pulmonary deposited dose based on terminal body weight with deposition
— Not calculated

### Discussion:

Inhaled delivery of ALTA-2530 achieved prolonged pulmonary exposure of rhIL-1Ra that exceeded 24 hr in rat compared to transient exposure of <20 min following bolus IV injection.

The prolonged exposure of rhIL-1Ra in lung following inhaled delivery of ALTA-2530 is predictive for once or twice daily, or less frequent, dosing clinically compared to multiple daily IV doses required for the treatment of lung pathologies.

The ratio of lung epithelial lining fluid to plasma exposures as AUC were >2500-fold across all inhaled doses compared to 0.44-fold for lung tissue: plasma following a 5 hr IV infusion (Kim et al, 1995).

IL-1Ra binds to the IL-1RI receptor with comparable affinity as IL-1b; thus, rhIL-1Ra levels ˜100X levels are needed for pharmacological levels in lung tissue. At human equivalent doses (based on mg/g lung) rat BALF rhIL-1ra concentrations exceeded those of IL-1b reported in BAL of BOS patients by >1000X.

Nebulized ALTA-2530 delivers stable and active rhIL-1Ra protein, in a particle size for delivery to small airways of the lung and exposure duration predictive for once daily therapeutic dosing in BOS.

Effective animal doses from in vivo studies (e.g., see Table 2 above) can be converted to appropriate human doses using conversion methods known in the art (e.g., see Tepper et al, Breathe in, breath out, it's easy: What you need to know about developing inhaled drugs“, Int J of Tox, 2016 35(4) 376-392). In some embodiments, the rat dose can be converted to human dose based on mg of ALTA-2530 per g of lung weight. In some embodiments, human patients are administered inhaled ALTA-2530 at doses of between about 0.5 mg/kg to about 2 mg/kg.

### Example 8—Assessing the Safety, Pharmacological and Biochemical Effects of Nebulized Anakinra in Healthy Smokers

An exploratory, single-dose, dose-escalating phase 1 study was performed with 18 healthy smokers. All 18 subjects received nebulized inhalation of anakinra. The subjects were separated into three (3) dose groups, and dosage forms of anakinra were administered as follows: six (6) subjects received a dosage level of 0.75 mg, six (6) subjects received a dosage level of 3.75 mg, and six (6) subjects received a dosage level of 7 mg. There was a 14-day interval between each successive dose group whereby the safety of four (4) subjects in the prior dose group was assessed. Safety assessment included physical examinations, vital sign measurements, clinical laboratory evaluations, documentation of AEs, electrocardiogram (ECG) assessments, and pulmonary function (FEV₁), forced expiratory flow (FEF) of 25-75%, and forced vital capacity (FVC). Bronchoscopy for pharmacologic and biochemical endpoint was performed on two (2) subjects within each dose group, after the four (4) subjects in each dose group were analyzed for safety. Bronchoscopic analysis was carried out independent of the safety analysis. The total duration of the study was 2.5 months.

### Example 9—Content Uniformity

This test is designed to demonstrate the uniformity of medication per spray (or minimum dose), consistent with the label claim, to be discharged from the actuator or mouthpiece, of an appropriate number (n=about 10 from beginning and n=about 10 from end) of containers from a batch. The primary purpose is to ensure spray content uniformity within the same container and among multiple containers of a batch. Techniques for thoroughly analyzing the spray discharged from the actuator or mouthpiece for the drug substance content include multiple sprays from beginning to the end of individual container, among containers, and among batches of drug product. This test provides an overall performance evaluation of a batch, assessing the formulation, the manufacturing process, and the pump. At most, two sprays per determination are used except in the case where the number of sprays per minimum dose specified in the product labeling is one. To ensure reproducible in vitro dose collection, the procedure will have controls for actuation parameters (e.g., stroke length, actuation force). The test is performed with units primed following the instructions in the labeling. The amount of drug substance delivered from the actuator or mouthpiece is expressed both as the actual amount and as a percentage of label claim.

The following acceptance criteria are used. However, alternative approaches (e.g., statistical) may be used to provide equal or greater assurance of spray content uniformity. In general, for acceptance of a batch (1) the amount of active ingredient per determination is not outside of 80 to 120 percent of label claim for more than 2 of 20 determinations (10 from beginning and 10 from end) from 10 containers, (2) none of the determinations is outside of 75 to 125 percent of the label claim, and (3) the mean for each of the beginning and end determinations are not outside of 85 to 115 percent of label claim.

1. A method for treating an inflammatory disorder of lower airways in a human subject in need thereof, comprising administering an effective amount of anakinra directly to the lower airways in the human subject; wherein the effective amount of anakinra is from about 0.1 mg to about 200 mg per day; and
wherein the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), idiopathic pulmonary fibrosis (IPF), pneumonitis, primary graft dysfunction (PGD), and a reperfusion injury.

2. The method of claim 1, wherein the toxic-inhalation lung injury is caused by inhalation of one or more toxic agents selected from the group consisting of a chemical warfare agent, an environmental toxic agent, and an industrial toxic agent.

3. The method of claim 2, wherein the chemical warfare agent is selected from the group consisting of chlorine gas and sulfur mustard.

4. (canceled)

5. The method of claim 2, wherein the toxic agent is selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes.

6. The method of claim 1, wherein the toxic-inhalation lung injury is pneumoconiosis or bronchiolitis obliterans, or a vaping-associated lung injury.

7. The method of claim 1, wherein the toxic-inhalation lung injury is chlorine-induced bronchiolitis obliterans syndrome (BOS) or sulfur mustard-induced bronchiolitis obliterans syndrome (BOS).

8. The method of claim 6, wherein the vaping-associated lung injury is caused by inhalation of one or more agents selected from the group consisting of diacetyl, α-Tocopheryl acetate, 2,3-pentanedione, nicotine, carbonyls, benzene, toluene, metals, bacterial endotoxins, and fungal glucans.

9. (canceled)

10. The method of claim 1, wherein the inflammatory disorder is an inflammatory disorder of the lung.

11. The method of claim 1, wherein the ARDS is associated with complications arising from viral infections caused by a virus selected from the group consisting of SARS-CoV-2, SARS-CoV, MERS-CoV, 229E, NL63, 0C43, and HKU1.

12-15. (canceled)

16. The method of claim 1, wherein the anakinra is administered in a pharmaceutical composition comprising anakinra and a pharmaceutically acceptable carrier.

17. The method of claim 16, wherein the pharmaceutically acceptable carrier comprises saline, Ringer's solution, dextrose solution, or a combination thereof.

18. (canceled)

19. The method of claim 1, wherein the effective amount of the anakinra is from about 0.1 mg to about 100 mg per day, from about 0.1 mg to about 50 mg per day, or from about 0.1 mg to about 10 mg per day.

20. The method of claim 19, wherein the effective amount of anakinra is from about 0.125 mg to 5.0 mg per day.

21.-141. (canceled)

142. The method of claim 1, wherein the inflammatory disorder is an inflammatory disorder of the upper airways of the lung.

143. The method of claim 16, where the pharmaceutically acceptable carrier comprises a buffer, a stabilizer, a tonicity modifier, or a combination thereof.

144. The method of claim 16, wherein a sustained exposure of the pharmaceutical composition in a lung epithelial lining fluid is between about 15 hours and about 100 hours.

145. The method of claim 144, wherein the sustained exposure of the pharmaceutical composition in the lung epithelial lining fluid is at least 24 hours.

146. The method of claim 16, wherein the pharmaceutical composition is administered between about once per week and about three times per day.

147. The method of claim 146, wherein the pharmaceutical composition is administered about once or twice daily.

148. The method of claim 16, wherein the pharmaceutical composition is administered via inhalation for between about 3 minutes and about 20 minutes.

149. The method of claim 16, wherein the anakinra in the pharmaceutical composition is administered at a dose of between about 0.5 mg/kg and about 2 mg/kg.

150. The method of claim 16, wherein the anakinra in the pharmaceutical composition binds with a substantially similar affinity as an endogenous IL-1β ligand to an IL-1 type 1 receptor.

151. The method of claim 1, wherein the anakinra is administered via inhalation or via direct instillation into the lower airways.

152. The method of claim 1, wherein the anakinra is administered by a delivery device selected from the group consisting of a nebulizer, an inhaler, and a subminiature aerolizer.

153. The method of claim 152, wherein the delivery device is a nebulizer.

154. The method of claim 153, wherein the delivery device is a mesh nebulizer.

155. The method of claim 154, wherein the nebulizer is configured to produce a droplet size of the liquid composition between about 5 μm and 30 μm in diameter.

156. The method of claim 155, wherein the nebulizer is configured to produce a droplet size of the liquid composition between about 0.5 μm and 10 μm in diameter.

157. The method of claim 156, wherein the nebulizer is configured to produce a droplet size of the liquid composition less than about 5 μm in diameter.

158. The method of claim 157, wherein the nebulizer is configured to produce a droplet size of the liquid composition less than about 3.5 μm in diameter.
